SEQUENCE LISTING (7 sequences)

SEQ ID NO: 1 - protein, 394 aa, Dengue virus
  source 1..394: note = serotype 1
RCVGIGNRDFVEGLSGGTWVDVVLEHGSCVTTMAKDKPTLDIELLKTEVTNPAVLRKLCIEAKISNTTTDSRCPTQGEATLVEEQDANFVCRRTFVDRGWGNGCGLFGKGSLITCAKFKCVTKLEGKIVQYENLKYSVIVTVHTGDQHQVGNETTEHGTIATITPQAPTSEIQLTDYGALTLDCSPRTGLDFDEMVLLTMKEKSWLVHKQWFLDLPLPWTSGASTPQETWNREDLLVTFKTAHAKKQEVVVLGSQEGAMHTALTGATEIQTSGTTKIFAGHLKCRLKMDKLTLKGMSYVMCTGSFKLEKEVAETQHGTVLVQVKYEGTDAPCKIPFSTQDEKGVTQNGRLITANPIVTDKEKPVNIEAEPPFGESYIVVGAGEKALKLSWFKKG

SEQ ID NO: 2 - protein, 394 aa, Dengue virus
  source 1..394: note = serotype 2
RCIGISNRDFVEGVSGGSWVDIVLEHGSCVTTMAKNKPTLDFELIKTEAKQSATLRKYCIEAKLTNTTTESRCPTQGEPSLNEEQDKRFVCKHSMVDRGWGNGCGLFGKGGIVTCAMFTCKKNMKGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKG

SEQ ID NO: 3 - protein, 390 aa, Dengue virus
  source 1..390: note = serotype 3
MRCVGVGNRDFVEGLSGATWVDVVLEHGGCVTTMAKNKPTLDIELQKTEATQPATLRKLCIEGKITNVTTDSRCPTQGEAILPEEQDQNYVCKHTYVDRGWGNGCGLFGKGSLVTCAKFQCLESIEGKAVQHENLKYTVIITVHTGDQHQVGNETQGVTAEITPQASTVEAILPEYGTLGLECSPRTGLDFNEMILLTMKNKAWMVHRQWFFDLPLPWTSGATTETPTWNKKELLVTFKNAHAKKQEVVVLGSQEGAMHTALTGATEIQTSGGTSIFAGHLKCRLKMDKLELKGMSYAMCLNAFVLKKEVSETQHGTILIKVEYKGEDAPCKIPFSTEDGQGKAHNGRLITANPIVTKKEEPVNIEAEPPFGESNIVIGIGDKALKINWY

SEQ ID NO: 4 - protein, 394 aa, Dengue virus
  source 1..394: note = serotype 4
RCVGVGNRDFVEGVSGGAWVDLVLEHGGCVTTMAQGKPTLDFELIKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDIPNHGVTATITPRSPSVEVKLPDYGELTLDCEPRSGIDFNEMILMKMKKKTWLVHKQWFLDLPLPWAAGADTSEVHWNYKERMVTFKVPHAKRQDVIVLGSQEGAMHSALTGATEVDSGDGNHMFAGHLKCKVRMEKLRIKGMSYTMCSGKFSIDKEMAETQHGTTVVKVKYEGAGAPCKVPIEIRDVNKEKVVGRIISSTPFAEYTNSVTNIELEPPFGDSYIVIGVGDSALTLHWFRKG

SEQ ID NO: 5 - protein, 495 aa, synthetic construct
  source 1..495: note = Chimeric dengue virus serotype 2
MRCIGMSNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFRCKKNMEGKVVQPENLEYTIVITPHSGEEHAVGNDTGKHGKEIKITPQSSITEAELTGYGTVTMECSPRTGLDFNEMVLLQMEDKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVTLVLVGIVTLYLGVMVQA

SEQ ID NO: 6 - protein, 352 aa, Dengue virus
  source 1..352: note = serotype 2 NS1 protein
DSGCVVSWKNKELKCGSGIFITDNVHTWTEQYKFQPESPSKLASAIQKAHEEGICGIRSVTRLENLMWKQITPELNHILSENEVKLTIMTGDIKGIMQAGKRSLRPQPTELKYSWKTWGKAKILSTEPHNQTFLIDGPETAECPNTNRAWNSLEVEDYGFGVFTTNIWLKLKERQDVFCDSKLMSAAIKDNRAVHADMGYWIESALNDTWKIEKASFIEVKSCHWPKSHTLWSNGVLESEMIIPKNFAGPVSQHNYRPGYHTQTAGPWHLGKLEMDFDFCEGTTVVVTEDCGNRGPSLRTTTASGKLITEWCCRSCTLPPLRYRGEDGCWYGMEIRPLKEKEENLVNSLVTA

SEQ ID NO: 7 - protein, 13 aa, Dengue virus
  source 1..13: note = native E protein fusion loop
DRGWGNGCGLFGK

(19) United States
(12) Patent Application Publication
(10) Pub. No.: US 2023/0366883 A1
(43) Pub. Date: Nov. 16, 2023

(54) Diagnostic Tests

(71) Applicants: Sanofi Pasteur, Lyon (FR); CTK Biotech, Inc., Poway, CA (US)

(72) Inventors: Yasemin Ataman-Onal, Reyrieux (FR); Matthew Bonaparte, Nazareth, PA (US); Catherine Chen, San Diego, CA (US); Vasco Liberal, Poway, CA (US); Qilin Pan, Temecula, CA (US); Stephen Savarino, Saylorsburg, PA (US); Wushan Yin, Beijing (CN); Lingyi Zheng, Bethlehem, PA (US)

(21) Appl. No.: 18/296,027

(22) Filed: Apr. 5, 2023

Related U.S. Application Data
(63) Continuation of application No. PCT/EP2021/077677, filed on Oct. 7, 2021.

(30) Foreign Application Priority Data
Mar. 30, 2021 (EP) 21315057.6

(51) Int. Cl. G01N 33/569; G01N 33/543; G01N 33/68

(52) U.S. Cl. CPC G01N 33/56983; G01N 33/54388; G01N 33/6857; G01N 2333/185; G01N 2469/20

(57) ABSTRACT

The present invention provides an immunodiagnostic test device for the detection of anti-dengue virus antibodies comprising a first dengue antigen and a second dengue antigen, wherein the first dengue antigen comprises a polypeptide having at least 90% sequence identity to SEQ ID NO. 1 and the second dengue antigen comprises a polypeptide having the sequence of SEQ ID NO. 2 or a polypeptide having a sequence which has at least 1 and no more than 4 amino acid substitutions with respect to the sequence of SEQ ID NO. 2.

### CROSS-REFERENCE TO RELATED APPLICATIONS

This patent application is a continuation of International Application No. PCT/EP2021/077677, filed on Oct. 7, 2021, which claims the benefit of priority to U.S. Provisional Application No. 63/088,564, filed Oct. 7, 2020, and European Application No. 21315057.6, filed Mar. 30, 3021, the contents of each of which are incorporated herein by reference in their entirety for all purposes.

### SEQUENCE LISTING

This patent application is filed with a sequence listing in electronic format. The Sequence Listing is provided as a file entitled “2023-03-30_01183-0280-00US-ABEL ST26.xml,” which was created on Mar. 30, 2023, and which is 10,522 bytes in size. The information in the electronic format of the sequence listing is incorporated herein by reference in its entirety.

### FIELD OF THE INVENTION

The present invention concerns diagnostic tests. More particularly, but not exclusively, this invention concerns dengue rapid diagnostic tests. The invention also concerns rapid diagnostic tests for identifying a prior dengue infection.

### BACKGROUND OF THE INVENTION

Dengue is the second most important infectious tropical disease after malaria with approximately one-half of the world's population living in areas where there is a risk of epidemic transmission. There are estimated to be 390 million cases of dengue every year and roughly 96 million people have clinically apparent disease. Each year, an estimated 500,000 people, including children, have a severe form of dengue requiring hospitalization, which puts a huge strain on health care systems during outbreaks. Approximately 2.5% of those affected with a severe form of dengue will die (World Health Organization. Dengue and dengue haemorrhagic fever, Fact sheet No 117, Updated May 2015. Available from URL: www.who.int/mediacentre/factsheets/fs117/en/.)

Dengue disease is caused by four antigenically distinct, but closely related dengue virus serotypes of the flavivirus genus (Gubler et al., 1988, in: Epidemiology of arthropod-borne viral disease. Monath T P M, editor, Boca Raton (FL): CRC Press: 223-60; Kautner et al., 1997, J. of Pediatrics, 131: 516-524; Rigau-Perez et al., 1998, Lancet, 352: 971-977; Vaughn et al., 1997, J. Infect. Dis., 176: 322-30). Dengue viruses are positive-sense, single-stranded RNA viruses.

Dengue disease is usually transmitted by injection of the dengue virus during the blood meal of an Aedes aegypti mosquito infected by the virus. After an incubation period of 4-10 days, the illness begins abruptly and is followed by three phases: febrile (2 to 7 days), critical (24-48 hours—during which severe complications may occur) and recovery (48-72 hours). During the critical phase, life threatening complications such as haemorrhages, shock and acute organ impairment may occur. A proper management of these unpredictable outcomes can reduce the case fatality rate. Cure of dengue fever is complete after 7 to 10 days, but prolonged asthenia is normal. Reduced leukocyte and platelet numbers are frequently observed.

Severe forms of dengue disease including dengue haemorrhagic fever (DHF) are potentially deadly complications of dengue virus infection. DHF is characterized by a high fever and symptoms of dengue disease, but with extreme lethargy and drowsiness. Increased vascular permeability and abnormal homeostasis can lead to a decrease in blood volume, hypotension, and in severe cases, hypovolemic shock and internal bleeding. Two factors appear to play a major role in the occurrence of DHF—rapid viral replication with a high level of viraemia (the severity of the disease being associated with the level of viraemia; Vaughn et al., 2000, J. Inf Dis., 181: 2-9) and a major inflammatory response with the release of high levels of inflammatory mediators (Rothman and Ennis, 1999, Virology, 257: 1-6; Alan L. Rothman. 2011, Nature Reviews Immunology, 11: 532-543). The mortality rate for DHF can reach 10% without treatment, but is <1% in most centres with access to treatment. Dengue disease infections are endemic in more than 100 tropical countries and DHF has been documented in 60 of these countries (Gubler, 2002, TRENDS in Microbiology, 10: 100-103).

Dengue shock syndrome (DSS) is a common progression of DHF and is frequently fatal. DSS results from generalized vasculitis leading to plasma leakage into the extravascular space. DSS is characterized by rapid and poor volume pulse, hypotension, cold extremities, and restlessness.

In Asia, DHF and DSS are observed primarily in children, with approximately 90% of those with DHF being less than 15 years of age (Malavige et al., 2004, Postgrad Med. J., 80: 588-601; Meulen et al., 2000, Trop. Med. Int. Health, 5:325-9). In contrast, outbreaks in the Caribbean and Central America have predominantly affected adults (Malavige et al., 2004, Postgrad Med. J., 80: 588-601). Incidence of dengue disease has increased in older age groups in many countries where dengue is endemic (Sabchareon et al, 2012, Lancet, 380, 1559-1567; Messina et al., 2014, Trends Microbiol., 22, 138-146).

The four serotypes of dengue virus possess approximately 60-80% sequence homology. Infection with one dengue serotype provides durable homologous immunity but limited heterologous immunity (Sabin, 1952, Am. J. Trop. Med. Hyg., 1: 30-50). Accordingly, an individual that has been infected with one serotype of dengue may subsequently become infected with a different serotype. It is considered that a second infection arising from a different dengue virus serotype is theoretically a risk factor for the development of severe dengue/DHF, since the majority of patients that exhibit severe dengue/DHF have been previously exposed to at least one of the other four serotypes of dengue virus.

To date, there is no specific treatment for dengue disease. Treatment for dengue disease is symptomatic, with bed rest, control of the fever and pain through antipyretics and analgesics, and adequate drinking. The treatment of DHF requires balancing of liquid losses, replacement of coagulation factors and the infusion of heparin.

Since dengue prevention measures, such as mosquito control and personal protection from bites are limited in efficacy, difficult to enforce and expensive, a safe and efficacious dengue vaccine is the best mode of prevention.

Sanofi Pasteur has previously developed a dengue vaccine, marketed under the commercial name DENGVAXIA®. The vaccine efficacy (VE) of this dengue vaccine was demonstrated inter alia in subjects from 2 to 16 years (Capeding M R, et al., 2014, Lancet; 384(9951):1358-65 and Villar L, et al., 2015, N Engl J Med., 372(2):113-23) and also in older subjects (aged 18-45) via the use of bridging methods based on neutralising antibody response (Gilbert P B, et al., 2019, Am J Trop Med Hyg, 101(1):164-179).

A close analysis of the results obtained with this dengue vaccine, however, shows that it is particularly effective in the protection against dengue disease of subjects who were already dengue seropositive at the time of the vaccination, i.e. subjects who had previously been infected by a dengue virus, irrespective of the serotype. For subjects initially dengue seronegative, i.e. who had not previously been infected by a dengue virus, the neutralizing antibody levels after the primary vaccination are, however, lower than the neutralizing antibody response generated in dengue seropositive subjects.

The impact of dengue serostatus was investigated in detail by Sridhar et al., (New Engl. J. Med., 2018, vol. 379: 327-340), which considered the effect of prior dengue serostatus (i.e., before vaccination) on the safety and efficacy of DENGVAXIA®. Sridhar found that the performance of the vaccine is superior in dengue seropositive subjects, for example, in vaccine recipients aged 9 to 16 years of age, statistically significant VE in seropositive subjects was 76%, compared to 39% in seronegative subjects. Furthermore, DENGVAXIA® was found to protect against severe dengue and hospitalisation for dengue for at least 5 years in subjects who were dengue seropositive before vaccination, but there was evidence of a higher risk of those outcomes in vaccinated subjects who were dengue seronegative.

Tests to determine dengue infections exist in the art. However, such tests are tailored to detect acute dengue, e.g., in a clinical setting, where a patient presents with a suspected case of dengue fever. Furthermore, such serotests lack the appropriate sensitivity and/or specificity leading to false positive or false negative results.

There is thus a need to develop a dengue serotest capable of identifying potential dengue vaccine subjects who are dengue seropositive with high specificity and sensitivity, in particular dengue seropositive subjects who have had a prior dengue infection at some time in the past (i.e., are not suffering from an acute dengue infection). Once identified those subjects can then be administered a dengue vaccine such as DENGVAXIA®.

The present invention seeks to address the above-mentioned need.

### SUMMARY OF THE INVENTION

The present invention provides an immunodiagnostic test device for the detection of anti-dengue virus antibodies comprising a first dengue antigen and a second dengue antigen, wherein said first dengue antigen comprises a polypeptide having at least 90% sequence identity to SEQ ID NO. 1 and said second dengue antigen comprises a polypeptide having the sequence of SEQ ID NO. 2 or a polypeptide having a sequence which has at least 1 and no more than 4 amino acid substitutions with respect to the sequence of SEQ ID NO. 2.

The present invention also provides an immunodiagnostic test device described herein for use in the diagnosis or for use as an aid in the diagnosis of a dengue infection in a human subject.

The present invention also provides a method for determining a dengue infection in a human subject, the method comprising:
a. providing a first dengue antigen and a second dengue antigen, wherein said first and second dengue antigens are as defined herein and wherein said dengue antigens are conjugated to a detectable moiety;
b. contacting the dengue antigens with a biological sample obtained from the subject, the biological sample potentially containing at least one binding partner capable of binding to at least one of the antigens; and
c. if said sample contains said at least one binding partner, detecting the presence of a complex that forms between at least one of the dengue antigen-detectable moiety conjugates and the at least one binding partner, which presence indicates a dengue virus infection in the subject.

The present invention also provides a method for identifying a dengue seropositive human subject comprising the steps of: (i) obtaining a biological sample from said subject and (ii) analysing said sample using an immunodiagnostic test device as defined herein.

The present invention also provides an in vitro method for detecting anti-dengue IgG antibodies in a human subject comprising the steps of: (i) obtaining a biological sample from said subject and (ii) analysing said sample using an immunodiagnostic test device as defined herein.

The present invention also provides a method of vaccinating a human subject against dengue comprising the steps of: (i) obtaining a biological sample from said subject; (ii) analysing said sample using an immunodiagnostic test device as defined herein in order to determine if said subject is dengue seropositive and (iii) if said human subject is dengue seropositive according to the analysis of step (ii), administering a dengue vaccine to said human subject.

It will of course be appreciated that features described in relation to one aspect of the present invention may be incorporated into other aspects of the present invention. For example, the method of the invention may incorporate any of the features described with reference to the test device of the invention and vice versa.

### DESCRIPTION OF THE DRAWINGS

FIG. 1A shows a classification algorithm which may be used to determine with high accuracy the dengue serostatus of samples obtained from human subjects.

FIG. 1B shows a calculation of 50% and 90% virus neutralisation in a PRNT assay. PRNT₅₀ is calculated using a 4-point linear regression with serum dilutions around the 50% neutralisation level compared to the virus input. PRNT₉₀ is calculated the same way using serum dilutions around the 90% neutralisation level.

FIG. 2A shows a schematic diagram of a lateral flow dengue diagnostic test (1) according to an embodiment of the invention. The test comprises a support (2) on which are overlaid a sample pad (3), a conjugate pad (4), an analytical pad (5) and an absorbent pad (6). The test further comprises a test line (7), a control line (8), dengue antigen conjugates (9) and control conjugates (10).

FIG. 2B shows an image of a dengue diagnostic test according to an embodiment of the invention, as it could be presented to the user. The test cassette comprises a sample application well (S), a buffer application well (B), a test line (T) and a control line (C).

### DEFINITIONS

The term “immunodiagnostic test device” as used herein, refers to a diagnostic test device that utilises the reaction of an antigen and a binding partner as its primary means of detection. The binding partner is a molecule that is able to specifically bind to the antigen and the term includes antibodies or fragments thereof such as Fab fragments, F(ab′)₂ fragments, Fv fragments etc. The immunodiagnostic test device is also referred to herein as the “test device” or simply the “device”.

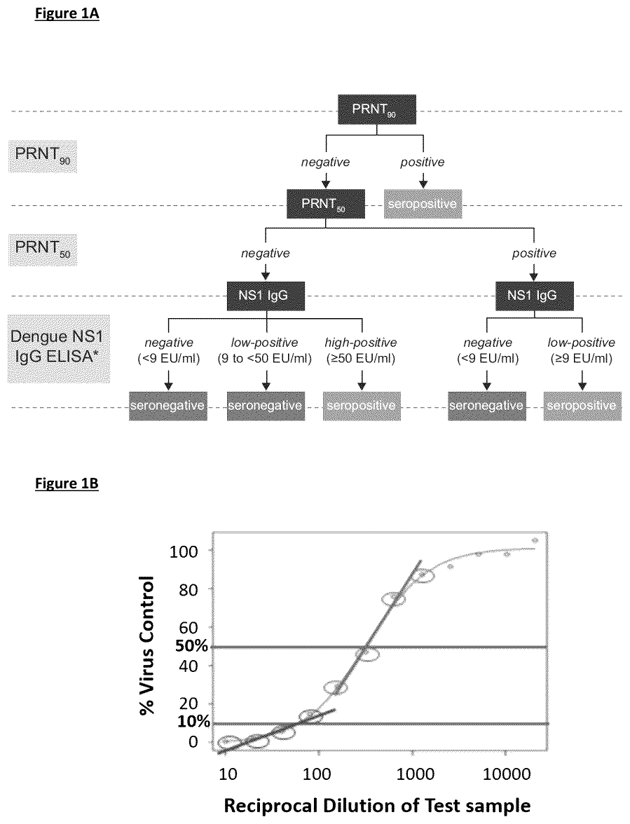

The term “dengue disease”, as used herein, refers to the clinical symptoms, of all grades of severity, exhibited by an individual following infection by a dengue virus. As used herein, the term dengue disease encompasses both the milder manifestations of dengue disease such as dengue fever and the more severe manifestations of dengue fever such as severe dengue or dengue haemorrhagic fever (DHF) as defined herein. Since 1975, clinical dengue has been classified according to World Health Organization guidelines (updated in 1997) as (i) dengue fever or (ii) dengue haemorrhagic fever (World Health Organization. Dengue hemorrhagic fever: Diagnosis, treatment, prevention and control 2nd Ed. Geneva: WHO, 1997; ISBN 92 4 154500 3). In 2009, the WHO issued new guidelines that classify clinical dengue as (i) dengue with or without warning signs or (ii) severe dengue. Both classifications are shown in FIGS. 1 & 2 of Srikiatkachorn et al., Clin. Infect. Dis. (2011) 53(6): 563. According to the earlier 1997 WHO classification, dengue fever is diagnosed by: (i) the presence of fever with at least two symptoms selected from headache, arthralgia, retro-orbital pain, rash, myalgia, haemorrhagic manifestations, and leucopenia; together with (ii) supportive serology or occurrence at the same location and time as other confirmed dengue cases. Progression to DHF is confirmed when fever, haemorrhagic manifestations, thrombocytopenia and evidence of plasma leakage are all observed. According to the 2009 WHO classification, diagnosis of dengue requires the presence of: (i) fever and at least two clinical symptoms selected from nausea, vomiting, rash, aches and pains, a positive tourniquet test, or any warning signs selected from abdominal pain and tenderness, persistent vomiting, clinical fluid accumulation, mucosal bleed, lethargy or restlessness, liver enlargement >2 cm or an increase in haematocrit concurrent with a rapid decrease in platelet count; together with (ii) supportive serology or occurrence at the same location and time as other confirmed dengue cases. According to the 2009 WHO classification, severe dengue is defined as a diagnosis of dengue with the observation of any of the following additional events: (i) severe plasma leakage leading to shock or respiratory distress (fluid accumulation); (ii) severe bleeding as evaluated by clinicians; or (iii) severe organ involvement (i.e. liver: AST, ALT ≥1000; CNS: impaired consciousness or heart or other organs).

The terms “Dengue haemorrhagic fever” or “DHF”, as used herein, are consistent with the 1997 WHO definition and refer to the following symptoms—1) Clinical manifestations: (a) Fever: acute onset, high (≥38° C.) and continuous lasting 2 to 7 days; (b) Any of the following haemorrhagic manifestations: a positive tourniquet test, petechiae, purpura, ecchymosis, epistaxis, gum bleeding, and hematemesis and/or melena; 2) Laboratory findings: (a) Thrombocytopenia (platelet count ≤100×109/L); (b) Plasma leakage as shown by haemoconcentration (haematocrit increased by 20% or more) or pleural effusion (seen on chest X-ray) and/or ascites and/or hypalbuminaemia. The first two clinical criteria (i.e. fever and haemorrhagic manifestations), plus thrombocytopenia and signs of plasma leakage are sufficient to establish a clinical diagnosis of DHF. Pleural effusion (seen on chest X-ray) and/or hypalbuminaemia provide supporting evidence of plasma leakage. DHF, as used herein, may be further defined on the basis of its severity. Thus DHF may be defined as being of Grade I, Grade II, Grade III or Grade IV (World Health Organization. Dengue hemorrhagic fever: Diagnosis, treatment, prevention and control 2nd Ed. Geneva: WHO, 1997; ISBN 92 4 154500 3). Grade I is defined as fever accompanied by non-specific constitutional symptoms; the only haemorrhagic manifestation is a positive tourniquet test. Grade II is defined as spontaneous bleeding in addition to the manifestations of Grade I patients, usually in the form of skin or other haemorrhages. Grade III is defined as circulatory failure manifested by a rapid, weak pulse and narrowing of pulse pressure (20 mmHg or less) or hypotension, with the presence of cold clammy skin and restlessness. Grade IV is defined as profound shock with undetectable blood pressure and pulse.

The term “virologically-confirmed dengue”, as used herein, refers to an acute febrile episode (i.e. temperature ≥38° C. on at least two consecutive days) which is confirmed to be induced by a dengue virus, e.g. by reverse transcriptase polymerase chain reaction (RT-PCR) and/or by a dengue non-structural 1 (NS1) protein enzyme-linked immunosorbent assay (ELISA). In the RT-PCR method, RNA is extracted from the serum to discard potential Taq polymerase inhibitors or interfering factors, using a commercial kit. Then a dengue screen RT-PCR reaction is carried out with primers from a gene sequence conserved among dengue viruses. Results are expressed as a concentration of log 10 plaque forming unit (PFU)/mL, by comparison with standards containing known concentrations of viral genomic nucleic acid sequences. Serotype identification of post-infectious dengue viremia is determined by testing serum samples with the Simplexa™ Dengue RT-PCR assay (Focus Diagnostics, Inc. CA, USA). Briefly, RNA is extracted from the serum to discard potential polymerase inhibitors or interfering factors, using a commercial kit. Then the Simplexa™ assay is carried out which incorporates serotype-specific primers from dengue sequences. The results are expressed qualitatively and reported for each dengue serotype as detected or not detected. The Simplexa™ assay is used on all dengue screen RT-PCR positive or dengue NS1 Ag ELISA positive samples for serotype identification. The NS1 ELISA is performed using a commercially available kit (Platelia™ Dengue NS1 Ag, Bio-Rad, Marnes-la-Coquette, France). The manufacturer's instructions are followed. The Dengue NS1 Ag test is a one-step sandwich-ELISA based assay that enables detection of NS1 Ag in serum. The test uses murine monoclonal Abs (MAbs) for capture and revelation. Samples and controls are directly and simultaneously incubated with the conjugate within the microplate wells coated with MAb. If NS1 Ag is present in the sample, an immune-complex MAb-NS1-MAb/peroxidase will be formed. The presence of immune-complex is demonstrated by addition of a chromogenic solution that initiates a colour development reaction. After 30 minutes of incubation at room temperature, the enzymatic reaction is stopped by addition of an acid solution. The optical density (OD) reading obtained with a spectrophotometer set at 450/620 nm is proportional to the amount of NS1 Ag present in the sample. The presence of NS1 Ag in an individual sample is determined by comparing the OD reading of the sample to the OD of the cut-off control serum. Sample ratios of <0.5, ≥0.5 to <1.0, and ≥1 are indicative of negative, equivocal, and positive results, respectively.

The terms “severe dengue” or “severe dengue disease”, as used herein refer to severe dengue defined as follows. In a case of dengue fever, the appearance of any one of the following criteria results in a diagnosis of severe dengue: (i) shock (pulse pressure ≤20 mmHg in a child or adolescent, or hypotension [≤90 mmHg] with tachycardia, weak pulse and poor perfusion); (ii) Bleeding requiring blood transfusion; (iii) Encephalopathy i.e., unconsciousness or poor conscious state or convulsions not attributable to simple febrile convulsion or focal neurological signs. Poor conscious state or unconsciousness must be supported by Glasgow Coma Scale (GCS) score; (iv) Liver impairment (AST >1000 U/L or prothrombin time [PT] International normalized ratio [INR]>1.5); (v) Impaired kidney function (Serum creatinine ≥1.5 mg/dL) or (vi) Myocarditis, pericarditis or heart failure (clinical heart failure) supported by chest X ray (CXR), echocardiography, electrocardiogram (ECG) or cardiac enzymes where these are available.

The terms “dengue fever virus” and “dengue virus” are used interchangeably. They refer to positive single-strand RNA viruses belonging to the Flavivirus genus of the family of flaviviridae. There are four different serotypes of dengue virus (serotypes 1, 2, 3 and 4), which possess approximately 60-80% sequence homology. The organization of the genome comprises the following elements: a 5′ non-coding region (NCR), a region encoding structural proteins (capsid (C), pre-membrane (prM) and envelope (E)) and a region encoding non-structural proteins (NS1-NS2A-NS2B-NS3-NS4A-NS4B-NS5) and a 3′ NCR. The dengue viral genome encodes an uninterrupted coding region which is translated into a single polyprotein which undergoes post-translational processing to generate the individual proteins.

As used herein, a “dengue immune” or “dengue seropositive” subject refers to a subject who has (previously) been infected by a dengue virus or immunized by a dengue vaccine. Thus, a sample (e.g. a serum sample) taken from said subject would produce a positive result in a suitable dengue IgG serotest, e.g. an immunodiagnostic test device of the present invention. An anti-dengue NS1 IgG ELISA as described herein can also be used to identify a dengue seropositive subject and, in particular, can be used to differentiate between subjects who have previously been (naturally) infected by a dengue virus and those subjects who have previously been immunized by a chimeric dengue vaccine in which the nucleic acid encoding the NS1 protein is not of dengue origin, e.g. DENGVAXIA® (Sanofi Pasteur, France). The nucleic acid encoding the NS1 protein in DENGVAXIA® is from yellow fever and thus DENGVAXIA® does not express dengue NS1. Accordingly, a subject who has been immunised with DENGVAXIA® would produce a negative result in an anti-dengue NS1 IgG ELISA. A dengue PRNT₅₀ as described herein could also be used to identify a dengue seropositive subject, although a PRNT cannot distinguish between prior natural dengue infection and dengue vaccination with DENGVAXIA®. The use of different dengue IgG serotests can be combined in order to identify dengue seropositive subjects. Preferably, a subject who is dengue seropositive, as referred to herein, is dengue seropositive due to natural dengue infection.

As used herein, a “dengue seronegative” (or “dengue naïve” or “dengue non-immune”) subject refers to a subject who has not been (previously) infected by a dengue virus nor previously immunized with a dengue vaccine. Thus a sample (e.g. a serum sample) taken from said subject would produce a negative result in a suitable dengue IgG serotest, e.g. an immunodiagnostic test device of the present invention.

As used herein, a “multitypic dengue immune” or “multitypic dengue seropositive” subject is defined as a subject, a serum sample from whom will produce a positive result (antibody titre >10) to at least two serotypes in a dengue PRNT₉₀.

As used herein, a “monotypic dengue immune” or “monotypic dengue seropositive” is defined as a subject, a serum sample from whom will produce a positive result (antibody titre >10) to only one serotype in a dengue PRNT₉₀.

### DETAILED DESCRIPTION

The present invention is directed to an improved immunodiagnostic test device for the detection of anti-dengue virus antibodies. In particular, the present invention is based on the surprising observation that an immunodiagnostic test device comprising two dengue antigens, i.e. a dengue antigen of serotype 1 and a dengue antigen of serotype 2 according to the present invention is capable of identifying dengue seropositive subjects, in particular dengue seropositive subjects who have had a prior dengue infection at some time in the past (i.e. are not suffering from an acute dengue infection) with high specificity and sensitivity. Since dengue vaccines may be safer and more effective in vaccine recipients who are baseline dengue seropositive (for example who have had a prior dengue infection), one purpose of the test device of the present invention is to reliably identify dengue seropositive subjects, who may then be administered a dengue vaccine. This may be referred to herein as a “test and vaccinate” or “screen and vaccinate” strategy.

Whilst a generalised increase in test specificity and sensitivity is obviously advantageous, the test device of the present invention offers particular advantages as follows. In particular, the test devices of the present invention show reduced or absent levels of cross-reactivity to other related flaviviruses (i.e. reduced or absent false positives). This improvement is particularly advantageous because false positive results obtained using a dengue diagnostic test (i.e. identifying a subject as dengue seropositive when they are in fact dengue seronegative) as part of a test and vaccinate strategy, would lead to subjects who are true dengue seronegatives receiving a dengue vaccine. Such subjects may then suffer from an increased risk of experiencing a case of severe dengue. The test devices of the present invention also have improved detection of monotypic immune subjects. The test devices of the present invention also have improved detection of prior dengue infections that occurred a long time in the past, e.g. more than 1 year or more than 5 years, for example 1-3 years, 1-5 years or 1-10 years.

The present invention relates to an immunodiagnostic test device for the detection of anti-dengue virus antibodies. In an embodiment of the invention, the test device comprises at least one dengue antigen as defined herein, for example 1, 2, 3, 4, 5 or 6 dengue antigens as defined herein. In an embodiment of the invention, the test device comprises 2 or more; 3 or more; or 4 or more dengue antigens as defined herein. In an embodiment of the invention, the test device comprises 2 dengue antigens, i.e. a first dengue antigen and a second dengue antigen as defined herein. In an embodiment of the invention, the test device comprises a dengue serotype 1 antigen and a dengue serotype 2 antigen as defined herein. In an embodiment of the invention, the test device comprises 4 dengue antigens, i.e. a first dengue antigen, a second dengue antigen, a third dengue antigen and a fourth dengue antigen as defined herein. In an embodiment of the invention, the test device comprises a dengue serotype 1 antigen, a dengue serotype 2 antigen, a dengue serotype 3 antigen and a dengue serotype 4 antigen as defined herein.

In a first aspect, the present invention provides an immunodiagnostic test device for the detection of anti-dengue virus antibodies comprising a first dengue antigen and a second dengue antigen, wherein said first dengue antigen comprises a polypeptide having at least 90% sequence identity to SEQ ID NO. 1 and said second dengue antigen comprises a polypeptide having the sequence of SEQ ID NO. 2 or a substituted polypeptide having a sequence which has at least 1 and no more than 4 amino acid substitutions with respect to the sequence of SEQ ID NO. 2.

In an embodiment of this aspect, said device further comprises a third dengue antigen or a fourth dengue antigen, wherein said third dengue antigen comprises a polypeptide having at least 90% sequence identity to SEQ ID NO. 3 and said fourth dengue antigen comprises a polypeptide having at least 90% sequence identity to SEQ ID NO. 4.

In an embodiment of this aspect, said device further comprises a third dengue antigen and a fourth dengue antigen, wherein said third dengue antigen comprises a polypeptide having at least 90% sequence identity to SEQ ID NO. 3 and said fourth dengue antigen comprises a polypeptide having at least 90% sequence identity to SEQ ID NO. 4.

In an embodiment of this aspect, amino acid substitutions are conservative amino acid substitutions. A conservative substitution is a substitution of one amino acid residue in a protein sequence for a different amino acid residue having similar biochemical properties. Typically, conservative substitutions have minimal or no impact on the activity of a resulting polypeptide. For example, an antigen containing one or more conservative amino acid substitutions usually retains the structure and function of the unmutated protein.

In an embodiment of this aspect, said first, second, third and/or fourth antigens are envelope proteins of the dengue virus.

In an embodiment of this aspect, said first, second, third and/or fourth antigens are monomeric envelope proteins of the dengue virus.

In an embodiment of this aspect, said first, second, third and/or fourth antigens are envelope proteins of the dengue virus which are not denatured.

In an embodiment of this aspect, said first, second, third and/or fourth antigens are monomeric dengue virus envelope proteins which are not denatured.

In an embodiment of this aspect, said first, second, third and/or fourth antigens are recombinant envelope proteins of the dengue virus.

In an embodiment of this aspect, said first, second, third and/or fourth antigens are recombinant monomeric envelope proteins of the dengue virus.

In an embodiment of this aspect, said first, second, third and/or fourth antigens are recombinant envelope proteins of the dengue virus which are not denatured.

In an embodiment of this aspect, said first, second, third and/or fourth antigens are recombinant monomeric envelope proteins of the dengue virus which are not denatured.

In an embodiment of this aspect, said second dengue antigen has been produced, i.e. expressed, in Drosophila Schneider 2 (S2) cells.

In an embodiment of this aspect, said second dengue antigen has not been derived from a viral lysate.

In an embodiment of this aspect, said first, second, third and fourth dengue antigens have been produced, i.e. expressed, in Drosophila Schneider 2 (S2) cells.

In an embodiment of this aspect, at least one of said dengue antigens comprises at least one epitope which is capable of binding to said anti-dengue virus antibodies. In an embodiment of this aspect, both of the dengue antigens or all four of the dengue antigens comprise at least one epitope which is capable of binding to said anti-dengue virus antibodies.

In an embodiment of this aspect, said anti-dengue virus antibodies are IgG antibodies, for example IgG antibodies which are capable of binding to at least one epitope on at least one of said dengue antigens.

In an embodiment of this aspect, said test device comprises no other dengue antigens beyond the combinations of dengue antigens as defined herein in each of the embodiments of the invention. For example, said test device does not comprise an additional dengue Envelope antigen, or, for example, said test device does not comprise a dengue NS1 antigen or, for example, said test device comprises neither an additional Envelope antigen nor an additional NS1 antigen.

In an embodiment of this aspect, said test device is capable only of detecting anti-dengue IgG antibodies, i.e. said test device cannot detect anti-dengue IgM antibodies.

In an embodiment of this aspect, said polypeptide of said first antigen does not comprise an amino acid substitution with respect to SEQ ID NO. 1 at the positions in the polypeptide which correspond to positions 75 and 76 of SEQ ID NO. 1.

In an embodiment of this aspect, said substituted polypeptide of said second antigen does not comprise an amino acid substitution with respect to SEQ ID NO. 2 at the positions in the polypeptide which correspond to positions 75 and 76 of SEQ ID NO. 2.

In an embodiment of this aspect, said polypeptide of said third antigen does not comprise an amino acid substitution with respect to SEQ ID NO. 3 at the positions in the polypeptide which correspond to positions 76 and 77 of SEQ ID NO. 3.

In an embodiment of this aspect, said polypeptide of said fourth antigen does not comprise an amino acid substitution with respect to SEQ ID NO. 4 at the positions in the polypeptide which correspond to positions 75 and 76 of SEQ ID NO. 4.

In an embodiment of this aspect, said polypeptide of said first antigen does not comprise an amino acid substitution with respect to SEQ ID NO. 1 at the positions in the polypeptide which correspond to positions 75 and 76 of SEQ ID NO. 1 and said substituted polypeptide of said second antigen does not comprise an amino acid substitution with respect to SEQ ID NO. 2 at the positions in the polypeptide which correspond to positions 75 and 76 of SEQ ID NO. 2.

In an embodiment of this aspect, said polypeptide of said first antigen does not comprise an amino acid substitution with respect to SEQ ID NO. 1 at the positions in the polypeptide which correspond to positions 75 and 76 of SEQ ID NO. 1; said substituted polypeptide of said second antigen does not comprise an amino acid substitution with respect to SEQ ID NO. 2 at the positions in the polypeptide which correspond to positions 75 and 76 of SEQ ID NO. 2; said polypeptide of said third antigen does not comprise an amino acid substitution with respect to SEQ ID NO. 3 at the positions in the polypeptide which correspond to positions 76 and 77 of SEQ ID NO. 3; and said polypeptide of said fourth antigen does not comprise an amino acid substitution with respect to SEQ ID NO. 4 at the positions in the polypeptide which correspond to positions 75 and 76 of SEQ ID NO. 4.

Throughout this description and claims, the sequence identity of a polypeptide to the identified sequences may be measured over the full length of the identified sequences.

In an embodiment of this aspect, said polypeptide of said first dengue antigen has at least 90% sequence identity to SEQ ID NO. 1, optionally wherein said polypeptide of said first dengue antigen does not comprise an amino acid substitution with respect to SEQ ID NO. 1 at the positions in the polypeptide which correspond to positions 97 to 109 of SEQ ID NO. 1, i.e. said polypeptide has the sequence DRGWGNGCGLFGK at the positions within the polypeptide which correspond to positions 97 to 109 of SEQ ID NO. 1.

In an embodiment of this aspect, said polypeptide of said first dengue antigen has at least 92% sequence identity to SEQ ID NO. 1.

In an embodiment of this aspect, said polypeptide of said first dengue antigen has at least 94% sequence identity to SEQ ID NO. 1.

In an embodiment of this aspect, said polypeptide of said first dengue antigen has at least 96% sequence identity to SEQ ID NO. 1.

In an embodiment of this aspect, said polypeptide of said first dengue antigen has at least 98% sequence identity to SEQ ID NO. 1.

In an embodiment of this aspect, said polypeptide of said first dengue antigen has at least 99% sequence identity to SEQ ID NO. 1.

In an embodiment of this aspect, said polypeptide of said first dengue antigen has the sequence of SEQ ID NO. 1 or has a sequence which has at least 1 and no more than 4 amino acid substitutions with respect to the sequence of SEQ ID NO. 1.

In an embodiment of this aspect, said polypeptide of said first dengue antigen has the sequence of SEQ ID NO. 1 or has a sequence which has at least 1 and no more than 3 amino acid substitutions with respect to the sequence of SEQ ID NO. 1.

In an embodiment of this aspect, said polypeptide of said first dengue antigen has the sequence of SEQ ID NO. 1 or has a sequence which has at least 1 and no more than 2 amino acid substitutions with respect to the sequence of SEQ ID NO. 1.

In an embodiment of this aspect, said polypeptide of said first dengue antigen has the sequence of SEQ ID NO. 1 or has a sequence which has 1 amino acid substitution with respect to the sequence of SEQ ID NO. 1.

In an embodiment of this aspect, said polypeptide of said first dengue antigen has at least 92% sequence identity to SEQ ID NO. 1, optionally wherein said polypeptide does not comprise an amino acid substitution with respect to SEQ ID NO. 1 at the positions in the polypeptide which correspond to positions 97 to 109 of SEQ ID NO. 1.

In an embodiment of this aspect, said polypeptide of said first dengue antigen has at least 94% sequence identity to SEQ ID NO. 1, optionally wherein said polypeptide does not comprise an amino acid substitution with respect to SEQ ID NO. 1 at the positions in the polypeptide which correspond to positions 97 to 109 of SEQ ID NO. 1.

In an embodiment of this aspect, said polypeptide of said first dengue antigen has at least 96% sequence identity to SEQ ID NO. 1, optionally wherein said polypeptide does not comprise an amino acid substitution with respect to SEQ ID NO. 1 at the positions in the polypeptide which correspond to positions 97 to 109 of SEQ ID NO. 1.

In an embodiment of this aspect, said polypeptide of said first dengue antigen has at least 98% sequence identity to SEQ ID NO. 1, optionally wherein said polypeptide does not comprise an amino acid substitution with respect to SEQ ID NO. 1 at the positions in the polypeptide which correspond to positions 97 to 109 of SEQ ID NO. 1.

In an embodiment of this aspect, said polypeptide of said first dengue antigen has at least 99% sequence identity to SEQ ID NO. 1, optionally wherein said polypeptide does not comprise an amino acid substitution with respect to SEQ ID NO. 1 at the positions in the polypeptide which correspond to positions 97 to 109 of SEQ ID NO. 1.

In an embodiment of this aspect, said polypeptide of said first dengue antigen has the sequence of SEQ ID NO. 1 or has a sequence which has at least 1 and no more than 4 amino acid substitutions with respect to the sequence of SEQ ID NO. 1, and which does not comprise an amino acid substitution with respect to SEQ ID NO. 1 at the positions in the polypeptide which correspond to positions 97 to 109 of SEQ ID NO. 1.

In an embodiment of this aspect, said polypeptide of said first dengue antigen has the sequence of SEQ ID NO. 1 or has a sequence which has at least 1 and no more than 3 amino acid substitutions with respect to the sequence of SEQ ID NO. 1, and which does not comprise an amino acid substitution with respect to SEQ ID NO. 1 at the positions in the polypeptide which correspond to positions 97 to 109 of SEQ ID NO. 1.

In an embodiment of this aspect, said polypeptide of said first dengue antigen has the sequence of SEQ ID NO. 1 or has a sequence which has at least 1 and no more than 2 amino acid substitutions with respect to the sequence of SEQ ID NO. 1, and which does not comprise an amino acid substitution with respect to SEQ ID NO. 1 at the positions in the polypeptide which correspond to positions 97 to 109 of SEQ ID NO. 1.

In an embodiment of this aspect, said polypeptide of said first dengue antigen has the sequence of SEQ ID NO. 1 or has a sequence which has 1 amino acid substitution with respect to the sequence of SEQ ID NO. 1, wherein the amino acid substitution with respect to SEQ ID NO. 1 is not at the positions in the polypeptide which correspond to positions 97 to 109 of SEQ ID NO. 1.

In an embodiment of this aspect, said polypeptide of said first dengue antigen has the sequence of SEQ ID NO. 1.

In an embodiment of this aspect, said polypeptide of said second dengue antigen has the sequence of SEQ ID NO. 2; or has a sequence which has at least 1 and no more than 4 amino acid substitutions with respect to the sequence of SEQ ID NO. 2, wherein said sequence does not comprise an amino acid substitution with respect to SEQ ID NO. 2 at the positions in the polypeptide which correspond to positions 5, 52, 119, 125 and 202 of SEQ ID NO. 2.

In an embodiment of this aspect, said polypeptide of said second dengue antigen has the sequence of SEQ ID NO. 2; or has a sequence which has at least 1 and no more than 3 amino acid substitutions with respect to the sequence of SEQ ID NO. 2, wherein said sequence does not comprise an amino acid substitution with respect to SEQ ID NO. 2 at the positions in the polypeptide which correspond to positions 5, 52, 119, 125 and 202 of SEQ ID NO. 2.

In an embodiment of this aspect, said polypeptide of said second dengue antigen has the sequence of SEQ ID NO. 2; or has a sequence which has at least 1 and no more than 2 amino acid substitutions with respect to the sequence of SEQ ID NO. 2, wherein said sequence does not comprise an amino acid substitution with respect to SEQ ID NO. 2 at the positions in the polypeptide which correspond to positions 5, 52, 119, 125 and 202 of SEQ ID NO. 2.

In an embodiment of this aspect, said polypeptide of said second dengue antigen has the sequence of SEQ ID NO. 2; or has a sequence which has 1 amino acid substitution with respect to the sequence of SEQ ID NO. 2, wherein said sequence does not comprise an amino acid substitution with respect to SEQ ID NO. 2 at the positions in the polypeptide which correspond to positions 5, 52, 119, 125 and 202 of SEQ ID NO. 2.

In an embodiment of this aspect, said polypeptide of said second dengue antigen has the sequence of SEQ ID NO. 2; or has a sequence which has at least 1 and no more than 4 amino acid substitutions with respect to the sequence of SEQ ID NO. 2, wherein said substituted polypeptide does not comprise an amino acid substitution with respect to SEQ ID NO. 2 at the positions in the polypeptide which correspond to positions 97 to 109 of SEQ ID NO. 2, i.e. said polypeptide has the sequence DRGWGNGCGLFGK at the positions within the polypeptide which correspond to positions 97 to 109 of SEQ ID NO. 2.

In an embodiment of this aspect, said polypeptide of said second dengue antigen has the sequence of SEQ ID NO. 2; or has a sequence which has at least 1 and no more than 3 amino acid substitutions with respect to the sequence of SEQ ID NO. 2, wherein said substituted polypeptide does not comprise an amino acid substitution with respect to SEQ ID NO. 2 at the positions in the polypeptide which correspond to positions 97 to 109 of SEQ ID NO. 2, i.e. said polypeptide has the sequence DRGWGNGCGLFGK at the positions within the polypeptide which correspond to positions 97 to 109 of SEQ ID NO. 2.

In an embodiment of this aspect, said polypeptide of said second dengue antigen has the sequence of SEQ ID NO. 2; or has a sequence which has at least 1 and no more than 2 amino acid substitutions with respect to the sequence of SEQ ID NO. 2, wherein said substituted polypeptide does not comprise an amino acid substitution with respect to SEQ ID NO. 2 at the positions in the polypeptide which correspond to positions 97 to 109 of SEQ ID NO. 2, i.e. said polypeptide has the sequence DRGWGNGCGLFGK at the positions within the polypeptide which correspond to positions 97 to 109 of SEQ ID NO. 2.

In an embodiment of this aspect, said polypeptide of said second dengue antigen has the sequence of SEQ ID NO. 2; or has a sequence which has 1 amino acid substitution with respect to the sequence of SEQ ID NO. 2, wherein the amino acid substitution with respect to SEQ ID NO. 2 is not at the positions in the polypeptide which correspond to positions 97 to 109 of SEQ ID NO. 2, i.e. said polypeptide has the sequence DRGWGNGCGLFGK at the positions within the polypeptide which correspond to positions 97 to 109 of SEQ ID NO. 2.

In an embodiment of this aspect, said polypeptide of said second dengue antigen has the sequence of SEQ ID NO. 2; or has a sequence which has at least 1 and no more than 4 amino acid substitutions with respect to the sequence of SEQ ID NO. 2, wherein said substituted polypeptide does not comprise an amino acid substitution with respect to SEQ ID NO. 2 at the positions in the polypeptide which correspond to positions 5, 52, 97 to 109, 119, 125 and 202 of SEQ ID NO. 2.

In an embodiment of this aspect, said polypeptide of said second dengue antigen has the sequence of SEQ ID NO. 2; or has a sequence which has at least 1 and no more than 3 amino acid substitutions with respect to the sequence of SEQ ID NO. 2, wherein said substituted polypeptide does not comprise an amino acid substitution with respect to SEQ ID NO. 2 at the positions in the polypeptide which correspond to positions 5, 52, 97 to 109, 119, 125 and 202 of SEQ ID NO. 2.

In an embodiment of this aspect, said polypeptide of said second dengue antigen has the sequence of SEQ ID NO. 2; or has a sequence which has at least 1 and no more than 2 amino acid substitutions with respect to the sequence of SEQ ID NO. 2, wherein said substituted polypeptide does not comprise an amino acid substitution with respect to SEQ ID NO. 2 at the positions in the polypeptide which correspond to positions 5, 52, 97 to 109, 119, 125 and 202 of SEQ ID NO. 2.

In an embodiment of this aspect, said polypeptide of said second dengue antigen has the sequence of SEQ ID NO. 2; or has a sequence which has 1 amino acid substitution with respect to the sequence of SEQ ID NO. 2, wherein the amino acid substitution with respect to SEQ ID NO. 2 is not at the positions in the polypeptide which correspond to positions 5, 52, 97 to 109, 119, 125 and 202 of SEQ ID NO. 2.

In an embodiment of this aspect, said second dengue antigen comprises a polypeptide having the sequence of SEQ ID NO. 2.

In an embodiment of this aspect, said immunodiagnostic test device for the detection of anti-dengue virus antibodies comprises a first dengue antigen and a second dengue antigen, wherein said first dengue antigen comprises a polypeptide having at least 90% sequence identity to SEQ ID NO. 1 and said second dengue antigen comprises a polypeptide having the sequence of SEQ ID NO. 2 or a substituted polypeptide having a sequence which has at least 1 and no more than 4 amino acid substitutions with respect to the sequence of SEQ ID NO. 2, and wherein said polypeptide of said first dengue antigen does not comprise an amino acid substitution with respect to SEQ ID NO. 1 at the positions in the polypeptide which correspond to positions 97 to 109 of SEQ ID NO. 1, i.e. said polypeptide has the sequence DRGWGNGCGLFGK at the positions within the polypeptide which correspond to positions 97 to 109 of SEQ ID NO. 1; and wherein substituted polypeptide of said second dengue antigen does not comprise an amino acid substitution with respect to SEQ ID NO. 2 at the positions in the polypeptide which correspond to positions 97 to 109 of SEQ ID NO. 2, i.e. said polypeptide has the sequence DRGWGNGCGLFGK at the positions within the polypeptide which correspond to positions 97 to 109 of SEQ ID NO. 2.

In an embodiment of this aspect, said third dengue antigen comprises a polypeptide having at least 90% sequence identity to SEQ ID NO. 3, and optionally does not comprise an amino acid substitution with respect to SEQ ID NO. 3 at the positions in the polypeptide which correspond to positions 98 to 110 of SEQ ID NO. 3, i.e. said polypeptide has the sequence DRGWGNGCGLFGK at the positions within the polypeptide which correspond to positions 98 to 110 of SEQ ID NO. 3.

In an embodiment of this aspect, said polypeptide of said third dengue antigen has at least 92% sequence identity to SEQ ID NO. 3.

In an embodiment of this aspect, said polypeptide of said third dengue antigen has at least 94% sequence identity to SEQ ID NO. 3.

In an embodiment of this aspect, said polypeptide of said third dengue antigen has at least 96% sequence identity to SEQ ID NO. 3.

In an embodiment of this aspect, said polypeptide of said third dengue antigen has at least 98% sequence identity to SEQ ID NO. 3.

In an embodiment of this aspect, said polypeptide of said third dengue antigen has at least 99% sequence identity to SEQ ID NO. 3.

In an embodiment of this aspect, said polypeptide of said third dengue antigen has the sequence of SEQ ID NO. 3 or has a sequence which has at least 1 and no more than 4 amino acid substitutions with respect to the sequence of SEQ ID NO. 3.

In an embodiment of this aspect, said polypeptide of said third dengue antigen has the sequence of SEQ ID NO. 3 or has a sequence which has at least 1 and no more than 3 amino acid substitutions with respect to the sequence of SEQ ID NO. 3.

In an embodiment of this aspect, said polypeptide of said third dengue antigen has the sequence of SEQ ID NO. 3 or has a sequence which has at least 1 and no more than 2 amino acid substitutions with respect to the sequence of SEQ ID NO. 3.

In an embodiment of this aspect, said third dengue antigen comprises a polypeptide having the sequence of SEQ ID NO. 3 or a polypeptide having a sequence which has 1 amino acid substitution with respect to the sequence of SEQ ID NO. 3.

In an embodiment of this aspect, said polypeptide of said third dengue antigen has at least 92% sequence identity to SEQ ID NO. 3, and does not comprise an amino acid substitution with respect to SEQ ID NO. 3 at the positions in the polypeptide which correspond to positions 98 to 110 of SEQ ID NO. 3.

In an embodiment of this aspect, said polypeptide of said third dengue antigen has at least 94% sequence identity to SEQ ID NO. 3, and does not comprise an amino acid substitution with respect to SEQ ID NO. 3 at the positions in the polypeptide which correspond to positions 98 to 110 of SEQ ID NO. 3.

In an embodiment of this aspect, said polypeptide of said third dengue antigen has at least 96% sequence identity to SEQ ID NO. 3, and does not comprise an amino acid substitution with respect to SEQ ID NO. 3 at the positions in the polypeptide which correspond to positions 98 to 110 of SEQ ID NO. 3.

In an embodiment of this aspect, said polypeptide of said third dengue antigen has at least 98% sequence identity to SEQ ID NO. 3, and does not comprise an amino acid substitution with respect to SEQ ID NO. 3 at the positions in the polypeptide which correspond to positions 98 to 110 of SEQ ID NO. 3.

In an embodiment of this aspect, said polypeptide of said third dengue antigen has at least 99% sequence identity to SEQ ID NO. 3, and does not comprise an amino acid substitution with respect to SEQ ID NO. 3 at the positions in the polypeptide which correspond to positions 98 to 110 of SEQ ID NO. 3.

In an embodiment of this aspect, said polypeptide of said third dengue antigen has the sequence of SEQ ID NO. 3 or has a sequence which has at least 1 and no more than 4 amino acid substitutions with respect to the sequence of SEQ ID NO. 3, wherein said substituted polypeptide does not comprise an amino acid substitution with respect to SEQ ID NO. 3 at the positions in the polypeptide which correspond to positions 98 to 110 of SEQ ID NO. 3.

In an embodiment of this aspect, said polypeptide of said third dengue antigen has the sequence of SEQ ID NO. 3 or has a sequence which has at least 1 and no more than 3 amino acid substitutions with respect to the sequence of SEQ ID NO. 3, wherein said substituted polypeptide does not comprise an amino acid substitution with respect to SEQ ID NO. 3 at the positions in the polypeptide which correspond to positions 98 to 110 of SEQ ID NO. 3.

In an embodiment of this aspect, said polypeptide of said third dengue antigen has the sequence of SEQ ID NO. 3 or has a sequence which has at least 1 and no more than 2 amino acid substitutions with respect to the sequence of SEQ ID NO. 3, wherein said substituted polypeptide does not comprise an amino acid substitution with respect to SEQ ID NO. 3 at the positions in the polypeptide which correspond to positions 98 to 110 of SEQ ID NO. 3.

In an embodiment of this aspect, said polypeptide of said third dengue antigen has the sequence of SEQ ID NO. 3 or has a sequence which has 1 substitution with respect to the sequence of SEQ ID NO. 3, wherein the amino acid substitution with respect to SEQ ID NO. 3 is not at the positions in the polypeptide which correspond to positions 98 to 110 of SEQ ID NO. 3.

In an embodiment of this aspect, said third dengue antigen comprises a polypeptide having the sequence of SEQ ID NO. 3.

In an embodiment of this aspect, said polypeptide of said fourth dengue antigen has at least 90% sequence identity to SEQ ID NO. 4, and optionally does not comprise an amino acid substitution with respect to SEQ ID NO. 4 at the positions in the polypeptide which correspond to positions 97 to 109 of SEQ ID NO. 4, i.e. said polypeptide has the sequence DRGWGNGCGLFGK at the positions within the polypeptide which correspond to positions 97 to 109 of SEQ ID NO. 4.

In an embodiment of this aspect, said polypeptide of said fourth dengue antigen has at least 92% sequence identity to SEQ ID NO. 4.

In an embodiment of this aspect, said polypeptide of said fourth dengue antigen has at least 94% sequence identity to SEQ ID NO. 4.

In an embodiment of this aspect, said polypeptide of said fourth dengue antigen has at least 96% sequence identity to SEQ ID NO. 4.

In an embodiment of this aspect, said polypeptide of said fourth dengue antigen has at least 98% sequence identity to SEQ ID NO. 4.

In an embodiment of this aspect, said polypeptide of said fourth dengue antigen has at least 99% sequence identity to SEQ ID NO. 4.

In an embodiment of this aspect, said polypeptide of said fourth dengue antigen has the sequence of SEQ ID NO. 4 or a sequence which has at least 1 and no more than 4 amino acid substitutions with respect to the sequence of SEQ ID NO. 4.

In an embodiment of this aspect, said polypeptide of said fourth dengue antigen has the sequence of SEQ ID NO. 4 or has sequence which has at least 1 and no more than 3 amino acid substitutions with respect to the sequence of SEQ ID NO. 4.

In an embodiment of this aspect, said polypeptide of said fourth dengue antigen has the sequence of SEQ ID NO. 4 or has a sequence which has at least 1 and no more than 2 amino acid substitutions with respect to the sequence of SEQ ID NO. 4.

In an embodiment of this aspect, said polypeptide of said fourth dengue antigen has the sequence of SEQ ID NO. 4 or has a sequence which has 1 amino acid substitution with respect to the sequence of SEQ ID NO. 4.

In an embodiment of this aspect, said polypeptide of said fourth dengue antigen has at least 92% sequence identity to SEQ ID NO. 4, and does not comprise an amino acid substitution with respect to SEQ ID NO. 4 at the positions in the polypeptide which correspond to positions 97 to 109 of SEQ ID NO. 4.

In an embodiment of this aspect, said polypeptide of said fourth dengue antigen has at least 94% sequence identity to SEQ ID NO. 4, and does not comprise an amino acid substitution with respect to SEQ ID NO. 4 at the positions in the polypeptide which correspond to positions 97 to 109 of SEQ ID NO. 4.

In an embodiment of this aspect, said polypeptide of said fourth dengue antigen has at least 96% sequence identity to SEQ ID NO. 4, and does not comprise an amino acid substitution with respect to SEQ ID NO. 4 at the positions in the polypeptide which correspond to positions 97 to 109 of SEQ ID NO. 4.

In an embodiment of this aspect, said polypeptide of said fourth dengue antigen has at least 98% sequence identity to SEQ ID NO. 4, and does not comprise an amino acid substitution with respect to SEQ ID NO. 4 at the positions in the polypeptide which correspond to positions 97 to 109 of SEQ ID NO. 4.

In an embodiment of this aspect, said polypeptide of said fourth dengue antigen has at least 99% sequence identity to SEQ ID NO. 4, and does not comprise an amino acid substitution with respect to SEQ ID NO. 4 at the positions in the polypeptide which correspond to positions 97 to 109 of SEQ ID NO. 4.

In an embodiment of this aspect, said polypeptide of said fourth dengue antigen has the sequence of SEQ ID NO. 4; or has a sequence which has at least 1 and no more than 4 amino acid substitutions with respect to the sequence of SEQ ID NO. 4, wherein said substituted polypeptide does not comprise an amino acid substitution with respect to SEQ ID NO. 4 at the positions in the polypeptide which correspond to positions 97 to 109 of SEQ ID NO. 4.

In an embodiment of this aspect, said polypeptide of said fourth dengue antigen has the sequence of SEQ ID NO. 4; or has a sequence which has at least 1 and no more than 3 amino acid substitutions with respect to the sequence of SEQ ID NO. 4, wherein said substituted polypeptide does not comprise an amino acid substitution with respect to SEQ ID NO. 4 at the positions in the polypeptide which correspond to positions 97 to 109 of SEQ ID NO. 4.

In an embodiment of this aspect, said polypeptide of said fourth dengue antigen has the sequence of SEQ ID NO. 4; or has a sequence which has at least 1 and no more than 2 amino acid substitutions with respect to the sequence of SEQ ID NO. 4, wherein said substituted polypeptide does not comprise an amino acid substitution with respect to SEQ ID NO. 4 at the positions in the polypeptide which correspond to positions 97 to 109 of SEQ ID NO. 4.

In an embodiment of this aspect, said polypeptide of said fourth dengue antigen has the sequence of SEQ ID NO. 4; or has a sequence which has 1 substitution with respect to the sequence of SEQ ID NO. 4, wherein the amino acid substitution with respect to SEQ ID NO. 4 is not at the positions in the polypeptide which correspond to positions 97 to 109 of SEQ ID NO. 4.

In an embodiment of this aspect, said fourth dengue antigen comprises a polypeptide having the sequence of SEQ ID NO. 4.

In an embodiment of this aspect, said immunodiagnostic test device for the detection of anti-dengue virus antibodies comprises a first dengue antigen and a second dengue antigen as defined herein, and said device further comprises a third dengue antigen and a fourth dengue antigen, wherein said third dengue antigen comprises a polypeptide having at least 90% sequence identity to SEQ ID NO. 3, and which does not comprise an amino acid substitution with respect to SEQ ID NO. 3 at the positions in the polypeptide which correspond to positions 98 to 110 of SEQ ID NO. 3, i.e. said polypeptide has the sequence DRGWGNGCGLFGK at the positions within the polypeptide which correspond to positions 98 to 110 of SEQ ID NO. 3; and wherein said fourth dengue antigen comprises a polypeptide having at least 90% sequence identity to SEQ ID NO. 4, and which does not comprise an amino acid substitution with respect to SEQ ID NO. 4 at the positions in the polypeptide which correspond to positions 97 to 109 of SEQ ID NO. 4, i.e. said polypeptide has the sequence DRGWGNGCGLFGK at the positions within the polypeptide which correspond to positions 97 to 109 of SEQ ID NO. 4.

In one embodiment of this aspect, the immunodiagnostic test device comprises a support, a sample pad, a conjugate pad, an analytical pad, an absorbent pad, at least one detection line and at least one quality control line. In this embodiment, the conjugate pad may comprise said dengue antigens and wherein each of said antigens are conjugated to a detectable moiety. Said detectable moiety may be, for example, a gold colloid. In this embodiment, at least one capture moiety may be immobilised at said detection line. Said capture moiety can be a monoclonal antibody, a polyclonal antibody or a mixture of different monoclonal antibodies or said capture moiety may be any other relevant binding partner with specificity for human IgG antibodies. In an embodiment of this aspect, said capture moiety is an antibody, for example an anti-human IgG monoclonal antibody. In another embodiment of this aspect, the conjugate pad may comprise control conjugates, which are capable of being immobilised at said quality control line. In this embodiment, said control conjugates comprise a detectable moiety, for example a gold colloid, and a binding partner, which is capable of binding to a complementary binding partner, wherein said complementary binding partner is immobilised at the quality control line. The binding partner may be, for example, a chicken IgY antibody and the complementary binding partner, which is immobilised at the quality control line, may be, for example, an anti-chicken IgY antibody, such as a goat anti-chicken IgY antibody.

In another aspect, the present invention provides the immunodiagnostic test device described herein for use in the diagnosis or for use as an aid in the diagnosis of a dengue infection in a subject.

In one embodiment of this aspect, the subject is a human. Accordingly, in this embodiment, the present invention provides the immunodiagnostic test device described herein for use in the diagnosis of a dengue infection in a human subject, e.g. a human who is aged at least 6 years old or at least 9 years old, e.g. who is aged 6-60 or 9-45 years old. In one embodiment of this aspect, the human subject resides in a dengue endemic area, e.g. an area where the rate of dengue seropositivity in the local population is at least 25%, at least 40%, at least 50%, at least 60%, at least 75%, at least 80% or at least 90%. In another embodiment of this aspect, the human subject resides in an area where the rate of dengue seropositivity in the local population is between 10% and 90%, for example between 10% and 80%, between 10% and 70%, between 10% and 60%, between 10% and 50%, between 20% and 80%, between 20% and 70%, between 20% and 60% or between 20% and 50%. The rate of dengue seropositivity for a local population may be indicated for the population as a whole or for a specific age group, for example 6 year olds, 9 year olds, 12 year olds, 15 year olds, 17 year olds, 19 year olds or 21 year olds.

In one embodiment of this aspect, said dengue infection is a prior dengue infection. A prior dengue infection is defined herein as an infection that has occurred in the past, i.e. it is not an acute dengue infection. An acute dengue infection in a subject may be characterised by the presence of fever in said subject and the presence of dengue virus and/or dengue NS1 protein and/or dengue virus RNA in a sample taken from said subject. In one embodiment of this aspect, a prior dengue infection occurred in said subject, e.g. said human subject, at least 28 days before the use of the immunodiagnostic test device of an embodiment of the invention in the diagnosis of dengue infection. In another embodiment, a prior dengue infection occurred in said subject, e.g. said human subject, at least 3 months, at least 6 months or at least 1 year, e.g. 1 year to 3 years, 1 year to 5 years or 1 year to 10 years before the use of the immunodiagnostic test device of an embodiment of the invention in the diagnosis of dengue infection. In one embodiment of this aspect, a prior dengue infection in a subject is characterised by the presence of anti-dengue IgG antibodies and the absence of dengue virus and/or dengue NS1 protein and/or dengue virus RNA in a sample taken from said subject.

In another aspect, the present invention provides a method for determining or detecting a dengue infection in a subject, the method comprising:
a. providing a first dengue antigen and a second dengue antigen, wherein said first and second dengue antigens are as defined herein, and wherein said dengue antigens are conjugated to a detectable moiety;
b. contacting the dengue antigens with a biological sample obtained from the subject, the biological sample potentially containing at least one binding partner capable of binding to at least one of the antigens; and
c. if said sample contains said at least one binding partner, detecting the presence of a complex that forms between at least one of the dengue antigen-detectable moiety conjugates and the at least one binding partner, which presence indicates a dengue virus infection in the subject.

In one embodiment of this aspect, the subject is a human, e.g. a human who is aged at least 6 years old or at least 9 years old, e.g. who is aged 6-60 or 9-45 years old. In another embodiment of this aspect, the human subject, e.g. a human who is aged at least 6 years old or at least 9 years old, e.g. who is aged 6-60 or 9-45 years old, resides in a dengue endemic area, e.g. an area where the rate of dengue seropositivity in the local population is at least 25%, at least 40%, at least 50%, at least 60%, at least 75%, at least 80% or at least 90%. In another embodiment of this aspect, the human subject, e.g. a human who is aged at least 6 years old or at least 9 years old, e.g. who is aged 6-60 or 9-45 years old, resides in an area where the rate of dengue seropositivity in the local population is between 10% and 90%, for example between 10% and 80%, between 10% and 70%, between 10% and 60%, between 10% and 50%, between 20% and 80%, between 20% and 70%, between 20% and 60% or between 20% and 50%. The rate of dengue seropositivity for a local population may be indicated for the population as a whole or for a specific age group, for example 6 year olds, 9 year olds, 12 year olds, 15 year olds, 17 year olds, 19 year olds or 21 year olds.

In one embodiment of this aspect, said step (a) further comprises providing a third dengue antigen and a fourth dengue antigen, wherein said third and fourth dengue antigens are as defined herein and wherein said dengue antigens are conjugated to a detectable moiety.

In one embodiment of this aspect, the method is an in vitro method.

In one embodiment of this aspect, the at least one binding partner is an antibody against dengue virus. In another embodiment of this aspect, the at least one binding partner is an IgG antibody against dengue virus.

In one embodiment of this aspect, the detectable moiety is a gold colloid.

In one embodiment of this aspect, the biological sample is selected from the group consisting of: saliva, whole blood (either venous or capillary (fingerstick) blood), plasma, serum, and urine.

In one embodiment of this aspect, the biological sample is selected from the group consisting of: whole blood (either venous or capillary (fingerstick) blood), plasma and serum.

In one embodiment of this aspect, the sample is whole blood (either venous or capillary (fingerstick)).

In one embodiment of this aspect, the sample is capillary whole blood.

In one embodiment of this aspect, the dengue infection is a prior dengue infection. A prior dengue infection is defined herein as an infection that has occurred in the past, i.e. it is not an acute dengue infection. An acute dengue infection in a subject may be characterised by the presence of fever in said subject and the presence of dengue virus and/or dengue NS1 protein in a sample taken from said subject. In one embodiment, a prior dengue infection has occurred in said subject, e.g. said human subject, at least 28 days before the biological sample is obtained from said subject. In another embodiment, a prior dengue infection has occurred in said subject, e.g. said human subject, at least 3 months, at least 6 months or at least 1 year, e.g. 1 year to 3 years, 1 year to 5 years or 1 year to 10 years before the biological sample is obtained from said subject. In one embodiment of this aspect, a prior dengue infection in a subject is characterised by the presence of anti-dengue IgG antibodies and the absence of dengue virus and/or dengue NS1 protein in a sample taken from said subject.

In one embodiment of this aspect, said step of detecting the presence of a complex that forms between at least one of the dengue antigen-detectable moiety conjugates and the at least one binding partner comprises contacting the complex with a molecule capable of hybridising to the at least one binding partner.

In one embodiment of this aspect, said molecule capable of hybridising to the at least one binding partner is an antibody with specificity for human antibodies.

In one embodiment of this aspect, said antibody with specificity for human antibodies is specific for human IgG antibodies.

In another aspect, the present invention provides a method for identifying a dengue seropositive subject comprising the steps of: (i) obtaining a biological sample from said subject and (ii) analysing said sample using an immunodiagnostic test device as defined herein.

In one embodiment of this aspect, the subject is a human, e.g., a human who is aged at least 6 years old or at least 9 years old, e.g. who is aged 6-60 or 9-45 years old. In another embodiment of this aspect, the human subject, e.g. a human who is aged at least 6 years old or at least 9 years old, e.g. who is aged 6-60 or 9-45 years old, resides in a dengue endemic area, e.g. an area where the rate of dengue seropositivity in the local population is at least 25%, at least 40%, at least 50%, at least 60%, at least 75%, at least 80% or at least 90%. In another embodiment of this aspect, the human subject, e.g. a human who is aged at least 6 years old or at least 9 years old, e.g. who is aged 6-60 or 9-45 years old, resides in an area where the rate of dengue seropositivity in the local population is between 10% and 90%, for example between 10% and 80%, between 10% and 70%, between 10% and 60%, between 10% and 50%, between 20% and 80%, between 20% and 70%, between 20% and 60% or between 20% and 50%. The rate of dengue seropositivity for a local population may be indicated for the population as a whole or for a specific age group, for example 6 year olds, 9 year olds, 12 year olds, 15 year olds, 17 year olds, 19 year olds or 21 year olds. In another embodiment of this aspect, the subject is a monotypic dengue seropositive subject, e.g. a human monotypic dengue seropositive subject. In another embodiment of this aspect, the subject is a multitypic dengue seropositive subject, e.g., a human multitypic dengue seropositive subject.

In another aspect, the present invention provides a method for identifying a dengue seronegative subject comprising the steps of: (i) obtaining a biological sample from said subject and (ii) analysing said sample using an immunodiagnostic test device as defined herein.

In one embodiment of this aspect, the subject is a human.

In another aspect, the present invention provides a method for detecting anti-dengue antibodies comprising the steps of: (i) obtaining a biological sample from said subject and (ii) analysing said sample using an immunodiagnostic test device as defined herein.

In one embodiment of this aspect, the method is an in vitro method.

In one embodiment of this aspect, the method is a method for detecting anti-dengue IgG antibodies, for example in a human subject, e.g. a human who is aged at least 6 years old or at least 9 years old, e.g. who is aged 6-60 or 9-45 years old. In another embodiment of this aspect, the human subject, e.g. a human who is aged at least 6 years old or at least 9 years old, e.g. who is aged 6-60 or 9-45 years old, resides in a dengue endemic area, e.g. an area where the rate of dengue seropositivity in the local population is at least 25%, at least 40%, at least 50%, at least 60%, at least 75%, at least 80% or at least 90%. In another embodiment of this aspect, the human subject, e.g. a human who is aged at least 6 years old or at least 9 years old, e.g. who is aged 6-60 or 9-45 years old, resides in an area where the rate of dengue seropositivity in the local population is between 10% and 90%, for example between 10% and 80%, between 10% and 70%, between 10% and 60%, between 10% and 50%, between 20% and 80%, between 20% and 70%, between 20% and 60% or between 20% and 50%. The rate of dengue seropositivity for a local population may be indicated for the population as a whole or for a specific age group, for example 6 year olds, 9 year olds, 12 year olds, 15 year olds, 17 year olds, 19 year olds or 21 year olds.

In one embodiment of this aspect, the method is an in vitro method for detecting anti-dengue IgG antibodies in a human subject comprising the steps of: (i) obtaining a biological sample from said subject and (ii) analysing said sample using an immunodiagnostic test device as defined herein.

In one embodiment of this aspect, the biological sample is selected from the group consisting of: saliva, whole blood (either venous or capillary (fingerstick) blood), plasma, serum, and urine.

In one embodiment of this aspect, the biological sample is selected from the group consisting of: whole blood (either venous or capillary (fingerstick) blood), plasma or serum.

In one embodiment of this aspect, the sample is whole blood (either venous or capillary (fingerstick) blood).

In one embodiment of this aspect, the sample is capillary whole blood.

In one embodiment of this aspect, the IgG antibodies are anti-dengue Envelope protein antibodies. In a particular embodiment of this aspect, the IgG antibodies are specific for at least one polypeptide sequence selected from the group consisting of SEQ ID NOs 1 to 4.

In another aspect, the invention provides a method of vaccinating a subject against dengue comprising the steps of: (i) obtaining a biological sample from said subject; (ii) analysing said sample using an immunodiagnostic test device as defined herein in order to determine if said subject is dengue seropositive and (iii) if said subject is dengue seropositive according to the analysis of step (ii), administering a dengue vaccine to said subject. Also in this aspect, the invention provides an immunodiagnostic test device as defined herein for use in a method of vaccinating a subject against dengue, wherein said method comprises the steps of: (i) obtaining a biological sample from said subject; (ii) analysing said sample using said immunodiagnostic test device as defined herein in order to determine if said subject is dengue seropositive and (iii) if said subject is dengue seropositive according to the analysis of step (ii), administering a dengue vaccine to said subject. Also in this aspect, the invention provides a method of vaccinating a subject against dengue, wherein said subject has previously been shown to be dengue seropositive using an immunodiagnostic test device as defined herein and wherein said method comprises the step of administering a dengue vaccine to said subject. Also in this aspect, the invention provides a dengue vaccine for use in a method of vaccinating a subject against dengue, wherein said subject has previously been shown to be dengue seropositive using an immunodiagnostic test device as defined herein.

In one embodiment of this aspect (i.e. the aspect of the invention which includes a step of vaccination as referred to in the preceding paragraph), the subject is a human, e.g. a human who is aged at least 6 years old or at least 9 years old, e.g. who is aged 6-60 or 9-45 years old. In another embodiment of this aspect, the human subject, e.g. a human who is aged at least 6 years old or at least 9 years old, e.g. who is aged 6-60 or 9-45 years old, resides in a dengue endemic area, e.g. an area where the rate of dengue seropositivity in the local population is at least 25%, at least 40%, at least 50%, at least 60%, at least 75%, at least 80% or at least 90%. In another embodiment of this aspect, the human subject, e.g. a human who is aged at least 6 years old or at least 9 years old, e.g. who is aged 6-60 or 9-45 years old, resides in an area where the rate of dengue seropositivity in the local population is between 10% and 90%, for example between 10% and 80%, between 10% and 70%, between 10% and 60%, between 10% and 50%, between 20% and 80%, between 20% and 70%, between 20% and 60% or between 20% and 50%. The rate of dengue seropositivity for a local population may be indicated for the population as a whole or for a specific age group, for example 6 year olds, 9 year olds, 12 year olds, 15 year olds, 17 year olds, 19 year olds or 21 year olds. In another embodiment of this aspect, the subject is a human who has not previously received a dengue vaccine. Thus, if a biological sample obtained from said subject is dengue seropositive according to the analysis of step (ii), dengue seropositivity would be the result of natural infection. In another embodiment of this aspect, the vaccine is a tetravalent vaccine which comprises a dengue antigen of each of serotypes 1 to 4. In another embodiment of this aspect, each of said dengue antigens of serotypes 1 to 4 is independently selected from the group consisting of a live attenuated (non-chimeric) dengue virus and a live attenuated chimeric dengue virus. In another embodiment of this aspect, each of said dengue antigens of serotypes 1 to 4 is a live attenuated chimeric yellow fever-dengue virus, for example the vaccine DENGVAXIA® (Sanofi Pasteur, Lyon, France). In another embodiment of this aspect, each of said dengue antigens of serotypes 1, 3 and 4 is a live attenuated chimeric dengue virus (specifically a chimeric dengue-dengue virus) and said dengue antigen of serotype 2 is a live attenuated non-chimeric dengue virus, for example the vaccine TAK-003 (Takeda Pharmaceutical Company Limited, Tokyo, Japan), see Biswal et al., The Lancet (2020), vol. 395, issue 10234, p. 1423-1433 (doi.org/10.1016/S0140-6736(20)30414-1). In another embodiment of this aspect, each of said dengue antigens of serotypes 1, 3 and 4 is a live attenuated non-chimeric dengue virus and said dengue antigen of serotype 2 is a live attenuated chimeric dengue virus, specifically a chimeric dengue-dengue virus, for example the vaccine TV003 (Butantan Institute, Brazil), see Kallas et al., The Lancet Infectious Diseases (2020), vol. 20(7), p. 839-850 (doi.org/10.1016/S1473-3099(20)30023-2). In another embodiment of this aspect, the present invention provides a dengue vaccine for use in a method of vaccinating a subject against dengue, wherein said subject has previously been shown to be dengue seropositive using an immunodiagnostic test device as defined herein and wherein said dengue vaccine is selected from the group consisting of: (i) a tetravalent vaccine comprising dengue antigens of each of serotypes 1 to 4, in which each of the dengue antigens of serotypes 1 to 4 is a live attenuated chimeric yellow fever-dengue virus (e.g. DENGVAXIA®); (ii) a tetravalent vaccine comprising dengue antigens of each of serotypes 1 to 4, in which each of the dengue antigens of serotypes 1, 3 and 4 is a live attenuated chimeric dengue virus (e.g. a chimeric dengue-dengue virus) and the dengue antigen of serotype 2 is a live attenuated non-chimeric dengue virus (e.g. TAK-003) and (iii) a tetravalent vaccine comprising dengue antigens of each of serotypes 1 to 4, in which each of said dengue antigens of serotypes 1, 3 and 4 is a live attenuated non-chimeric dengue virus and said dengue antigen of serotype 2 is a live attenuated chimeric dengue virus (e.g. a chimeric dengue-dengue virus), for example the vaccine TV003. The subject who is vaccinated against dengue is preferably a human, e.g., a human who is aged at least 6 years old or at least 9 years old, e.g., who is aged 6-60 or 9-45 years old. In another embodiment of this aspect, the human subject, e.g. a human who is aged at least 6 years old or at least 9 years old, e.g. who is aged 6-60 or 9-45 years old, resides in a dengue endemic area, e.g. an area where the rate of dengue seropositivity in the local population is at least 25%, at least 40%, at least 50%, at least 60%, at least 75%, at least 80% or at least 90%. In another embodiment of this aspect, the human subject, e.g. a human who is aged at least 6 years old or at least 9 years old, e.g. who is aged 6-60 or 9-45 years old, resides in an area where the rate of dengue seropositivity in the local population is between 10% and 90%, for example between 10% and 80%, between 10% and 70%, between 10% and 60%, between 10% and 50%, between 20% and 80%, between 20% and 70%, between 20% and 60% or between 20% and 50%. The rate of dengue seropositivity for a local population may be indicated for the population as a whole or for a specific age group, for example 6 year olds, 9 year olds, 12 year olds, 15 year olds, 17 year olds, 19 year olds or 21 year olds.

In another aspect, the present invention further provides a method of manufacturing any of the immunodiagnostic test devices as described herein. For example, the present invention provides a method of manufacturing an immunodiagnostic test device comprising a support, a sample application zone, an antigen conjugate zone comprising at least one conjugated dengue antigen as described herein, an analytical zone comprising an immobilised capture moiety and an absorbent zone, wherein said method comprises the steps of: (i) providing said support; (ii) affixing said capture zone, absorbent zone, antigen conjugate zone and sample application zone to said support, thus forming said immunodiagnostic test device. In an embodiment of this aspect, said antigen conjugate zone comprises a first conjugated dengue antigen and a second conjugated dengue antigen, wherein said first and said second dengue antigens are as described herein. In an embodiment of this aspect, said antigen conjugate zone comprises a first conjugated dengue antigen, a second conjugated dengue antigen, a third conjugated dengue antigen and a fourth conjugated dengue antigen, wherein said first, second, third and fourth dengue antigens are as described herein. In an embodiment of this aspect, said conjugated dengue antigens are conjugated to a detectable moiety, e.g. a gold colloid. In an embodiment of this aspect, said immobilised capture moiety is a monoclonal antibody, e.g. an anti-human IgG antibody. In an embodiment of this aspect, said affixing of said capture zone, absorbent zone, antigen conjugate zone and sample application zone to said support is achieved via lamination. In an embodiment of this aspect, said method further comprises a step of affixing a control capture moiety, e.g. an anti-chicken IgY antibody, to said support and including a control conjugate as defined herein in said antigen zone. In an embodiment of this aspect, said support is nitrocellulose.

The Examples of the present invention describe an analysis of the ability of an immunodiagnostic test of the present invention to distinguish between serum samples which are seropositive or seronegative for dengue and other related flaviviruses such as Japanese Encephalitis Virus (JEV), West Nile Virus (WNV) and Yellow Fever Virus (YFV). In order to correctly investigate the properties of an immunodiagnostic test of the present invention, it is necessary to determine with high certainty the actual flavivirus status of the serum samples using other validated flavivirus serotests. Methods by which the flavivirus status of serum samples may be determined are as described in the following paragraphs.

If a serum sample has been taken from a subject residing in a dengue non-endemic area (e.g., the USA, mainland Europe etc.), a suitable serotest for determining whether the sample is dengue seropositive or dengue seronegative would be a dengue Plaque Reduction Neutralization Test (PRNT₅₀) assay as described in Timiryasova, T. M. et al., Am. J. Trop. Med. Hyg. (2013), vol. 88(5), 962-970. Briefly, serial, two-fold dilutions of the sera (previously heat-inactivated) are mixed with a constant challenge-dose of each dengue virus of serotype 1, 2, 3 or 4 as appropriate (expressed as PFU/mL). The parental dengue virus strains of the DENGVAXIA® dengue vaccine constructs may be used as the challenge strains. Those parental strains are as follows—serotype 1: strain PU0359 (Guirakhoo et al., J. Virol., (2001) vol. 75(16): 7290-7304 and GenBank sequence record AF425630); serotype 2: strain PU0218 (Gruenberg et al., J. Gen. Virol. (1988), vol. 69:1391-1398 and GenBank sequence record D00345); serotype 3: strain PaH881/88 (Guirakhoo et al., J. Virol., (2001) vol. 75(16): 7290-7304 and GenBank sequence record AF349753) and serotype 4: strain 1228 (Guirakhoo et al., J. Virol., (2001) vol. 75(16): 7290-7304 and GenBank sequence record JN022608). The mixtures are then inoculated into wells of a microplate with confluent Vero cell monolayers (CC1-81 cells, available from the ATCC (Manassas, VA, USA). After adsorption, cell monolayers are incubated for a few days. The presence of dengue virus infected cells is indicated by the formation of infected foci (i.e., plaques) and a reduction in virus infectivity due to the presence of neutralising antibodies in the serum samples (i.e. a reduction in the number of plaques) can thus be detected. The reported value (end point neutralization titre) represents the highest dilution of serum at which ≥50% of dengue challenge virus (in plaque counts) is neutralized when compared to the mean viral plaque count in the negative control wells (which represents the 100% virus load). The end point neutralization titres are presented as continuous values. The lower limit of quantification (LLOQ) of the assay is 10 (1/dil). It has been commonly considered that seroconversion occurs when the titre is superior or equal to 10 (1/dil). As PRNT tests may slightly vary from a laboratory to another the LLOQ may also slightly vary. Accordingly, in a general manner, it is considered that seroconversion occurs when the titre is superior or equal to the LLOQ of the test. However, as an alternative, a higher cut-off for determining seroconversion (i.e., a positive result) may be used in the context of the PRNT₅₀, for example, 25 (1/dil), 50 (1/dil), 75 (1/dil) or 100 (1/dil).

If the serum sample is taken from a subject residing in a dengue endemic area (e.g. in Brazil, Colombia, Thailand, the Philippines etc.), wherein said sample may thus contain antibodies specific for other related flaviviruses, the dengue serostatus of the sample is preferably determined according to the classification algorithm shown in FIG. 1A, which combines the use of three dengue serotests, namely a PRNT₉₀ assay, a PRNT₅₀ assay and an anti-dengue NS1 IgG ELISA. The higher cut off value of 90% neutralisation for the PRNT₉₀ assay can be recalculated from the original plaque counts obtained in the testing for the 50% neutralisation (see FIG. 1B). Thus PRNT₅₀ is calculated using a 4-point linear regression with serum dilutions around the 50% neutralisation level compared to the virus input and PRNT₉₀ is calculated in the same way using serum dilutions around the 90% neutralisation level. The value of the algorithm of FIG. 1A in assessing serum samples from subjects residing in dengue endemic areas is as follows. As described above, the dengue PRNT is a functional assay based on virus neutralization. Positivity for PRNT₅₀ and PRNT₉₀ is defined for samples with titers ≥10 (1/dilution [dil]) against at least one dengue serotype. According to the WHO guidelines on dengue PRNT testing (World Health Organization—Guidelines for plaque reduction neutralization testing of human antibodies to dengue viruses. Geneva: WHO/IVB/07.07; 2007), the PRNT assay is the most serologically virus-specific test among flaviviruses and serotype-specific test among dengue viruses. The guidelines state that PRNT₉₀ is more useful than PRNT₅₀ for evaluating previous dengue exposure in endemic areas, since it decreases the background of cross-reactive neutralizing antibodies against related flaviviruses and results in higher specificity. However, tests with very high specificity are likely to have lower sensitivity than less specific tests. As a result, some samples that test dengue negative in the PRNT₉₀ assay may be truly dengue positive (PRNT₉₀ false negatives). To minimize the risk of false negatives, the classification algorithm shown in FIG. 1A introduces information from two additional serotests (PRNT₅₀ and an anti-dengue NS1 ELISA), in an attempt to “rescue” the PRNT₉₀ false negatives as dengue positives, whilst at the same time maintaining minimal false dengue positivity. In respect of the PRNT, the primary viral component thought to play a role in dengue virus antibody neutralization in flaviviruses is the Envelope protein while the pre-Membrane protein plays a limited role. In contrast, NS1 is not a constituent of the virion but rather is secreted into the circulation by infected host cells during acute infection. The dengue NS1 IgG ELISA is considered useful precisely because the NS1 protein is not well conserved across flaviviruses. Initial characterization studies indicated that a threshold level of 9 ELISA units (EU)/mL was associated with very high sensitivity (>95%, low false dengue seronegativity), and a threshold of 50 EU/mL was associated with very high specificity in identifying dengue seropositive individuals. This was further confirmed in a large set of samples, which revealed that >99% of samples with a dengue NS1 IgG ELISA readout of ≥50 EU/ml were also PRNT₉₀ positive (Nascimento E J M et al., J. Virol. Methods 2018; 257:48-57). Since the main viral targets in the PRNT assay are different from the target in the dengue NS1 IgG assay, these assays provide complementary information about the likelihood of previous dengue exposure, leading to the classification algorithm shown in FIG. 1A.

The dengue anti-NS1 IgG ELISA was performed as described in Nascimento E J M et al., J. Virol. Methods 2018; 257:48-57 (doi.org/10.1016/j.jviromet.2018.03.007). Briefly, equimolar concentrations of all 4 serotypes of Dengue NS1 antigen (Native Antigen Company, Oxfordshire, UK) were coated onto a 96-well microtiter plate in carbonate/bicarbonate buffer pH 9.6±0.1 overnight at 4° C. Coated plates were washed with 0.01 M phosphate buffered saline+0.05% Tween 20 (PBS-T; Hyclone, Logan, USA) and blocked with PBS-T supplemented with 1% (v/v) goat normal serum (1% GNS; Gibco, Gaithersburg, USA) for 45±5 minutes at 21° C. The plates were washed again with PBS-T, then 2-fold serially-diluted human samples and internal quality controls in 1% GNS were added and incubated for 60±5 minutes at 37° C. Plates were washed a third time with PBS-T and incubated with peroxidase-conjugated F(ab′)₂ goat anti-human IgG, Fcγ Fragment (Jackson Immuno Research, West Grove, USA) in 1% GNS for 60±5 minutes at 37° C. The plates were washed again for the fourth time with PBS-T and colour was developed with SureBlue Reserve TMB Microwell Peroxidase Substrate (SeraCare, Milford, USA) for 30±2 minutes at 21° C. The reaction was stopped with 1 N HCl (Fisher Scientific, Fair Lawn, USA) and optical density at 450 nm (650 nm as the reference wavelength) measured using a SpectraMax Plus 384 Microplate reader (Molecular Devices, Sunnyvale, USA) in conjunction with SoftMax Pro software version 6.5.1 (Molecular Devices, Sunnyvale, USA). The geometric mean concentration (GMC) of anti-dengue NS1 IgG in the serum samples was determined in relation to the reference standards using a 4-parameter logistic (4 PL) model built into the SoftMax Pro Software using a minimum of 3 values, corrected for sample dilution to determine the concentration of each sample reported in ELISA Units per millilitre (EU/mL).

Japanese Encephalitis virus neutralizing antibody measurement may be assessed by PRNT. Serial 10-fold dilutions of serum to be tested (previously heat-inactivated) are mixed with a constant challenge dose (expressed as PFU/mL) of the JE Beijing strain (genotype III). The mixtures are inoculated into wells of a plate of confluent LLC-MK2 cells. After adsorption, cell monolayers are overlaid and incubated for several days. The reported value (end point neutralization titre) represents the highest dilution of serum at which ≥50% of JE challenge virus is neutralized when compared to the negative control wells, which represents the 100% virus load.

Yellow Fever neutralizing antibody levels may be measured by PRNT. Briefly, serial two-fold dilutions of serum to be tested (previously heat-inactivated) are mixed with a constant concentration of the YF vaccinal strain 17D (Theiler M. and Smith H. H., 1937, J. Exp. Med., 65. 767-786), expressed as PFU/mL. The mixtures are inoculated in duplicate into wells of a plate of confluent Vero cells. After adsorption, cell monolayers are overlaid and incubated for a few days. The reported value (end point neutralization titre) represents the highest dilution of serum at which ≥50% of YF challenge virus (in plaque counts) is neutralized when compared to the negative control wells, which represents the 100% virus load.

Zika neutralising antibodies may be assessed by a microneutralisation assay as described in Nascimento E J M et al., Am. J. Trop. Med. Hyg., 101(3), 2019, pp. 708-715 (doi:10.4269/ajtmh.19-0270). Briefly, serial, two-fold dilutions of serum to be tested (previously heat-inactivated) are mixed with a constant concentration of Zika virus PRVABC59 (VR-1843, American Type Culture Collection [ATCC], Rockville, MD). The mixtures are inoculated in duplicate into wells of a 96-well microplate with permissive cells. After adsorption, cell monolayers are incubated for a few days. A reduction in virus infectivity (viral antigen production) due to neutralization by antibody present in serum samples is detected by ELISA. After washing and fixation, Zika viral antigen production in cells is detected by successive incubations with Zika-specific monoclonal antibodies (anti-pan Flavivirus mAb, HB112-4G2 from Biotem Inc., Apprieu, France), anti-mouse Ig conjugate (horseradish-conjugated goat anti-mouse IgG from Jackson ImmunoResearch Laboratories), and a chromogenic substrate (TMB microwell peroxidase substrate system from SeraCare). The resulting OD is measured using a microplate reader. The reduction in Zika virus infectivity as compared to that in the virus control wells constitutes a positive neutralization reaction indicating the presence of neutralizing antibodies in the serum sample. On the other hand, the infection of cells indicates the absence of neutralizing antibodies in the serum sample.

When using an immunodiagnostic test device as disclosed herein, e.g. in the context of a method as disclosed herein, it may be preferable to also test the subject for Severe acute respiratory syndrome coronavirus 2 (SARS-CoV-2), the causative agent behind coronavirus disease 2019 (COVID-19), to ensure that the subject is not also positive for SARS-CoV-2. Many tests exist to determine SARS-CoV-2 positivity, for example the OnSite COVID-19 Ag Rapid Test or the Aridia COVID-19 Real-Time PCR Test, both manufactured by CTK Biotech (CA, USA). Furthermore, when using an immunodiagnostic test device as disclosed herein, e.g. in the context of a method as disclosed herein, it may be preferable to avoid using the test in the context of the acute phase of a SARS-CoV-2 outbreak, e.g. when using an immunodiagnostic test device as disclosed herein, it may be preferable to use the test in areas where SARS-CoV-2 is not (actively) circulating and/or there are not likely to be acute SARS-CoV-2 infections.

Percent identity between two amino acid sequences or two nucleotide sequences may be determined, for example by manual alignment of the two sequences. Alternatively, percentage identity may be more conveniently determined by standard alignment algorithms such as, for example, Basic Local Alignment Tool (BLAST) described in Altschul et al. (1990) J. Mol. Biol., 215: 403-410; the algorithm of Needleman et al. (1970) J. Mol. Biol., 48: 444-453; the algorithm of Meyers et al. (1988) Comput. Appl. Biosci., 4: 1 1-17; or Tatusova et al. (1999) FEMS Microbiol. Lett., 174: 247-250, etc. Such algorithms are incorporated into the Nucleotide BLAST and the Protein BLAST programs (see https://blast.ncbi.nlm.nih.gov/Blast.cgi). When utilizing such programs, the default parameters can be used. For example, for nucleotide sequences the following settings can be used for Nucleotide BLAST: expect threshold of 0.05; word size of 28; maximum matches in a query range of 0; match/mismatch scores of 1, −2 and linear gap costs. For amino acid sequences the following settings can be used for Protein BLAST: expect threshold of 0.05; word size of 3; maximum matches in a query range of 0; matrix BLOSUM62; gap costs—existence 11 and extension 1 and a conditional compositional score matrix adjustment.

### EXAMPLES

### Example 1—Structure, Construction and Operation of an Immunodiagnostic Test Device

An immunodiagnostic test device of the present invention may be a lateral flow diagnostic test, otherwise known as rapid diagnostic test (RDT). The following example provides a description of the structure, construction and operation of such a test.

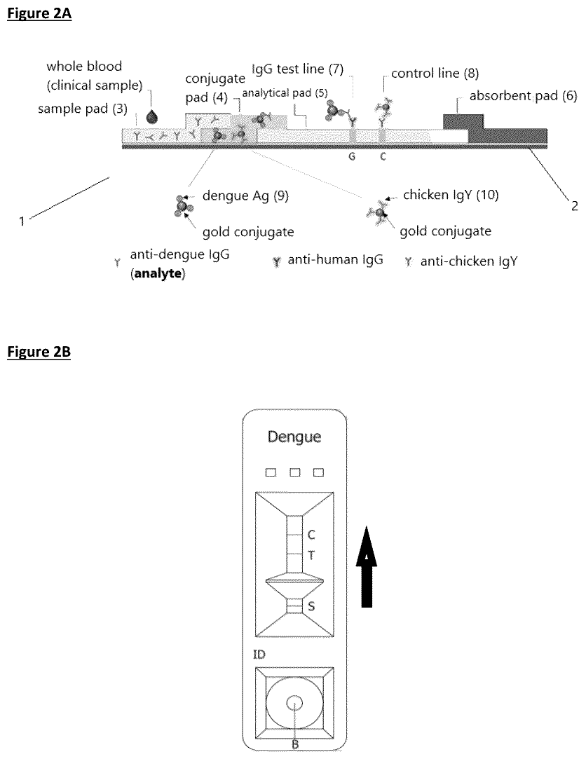

A schematic diagram of a lateral flow dengue diagnostic test according to an embodiment of the present invention is as shown in FIG. 2A. FIG. 2B shows a diagram of a lateral flow dengue diagnostic test according to an embodiment of the present invention as could be presented to the end user. As can be seen from FIG. 2A, the test comprises a nitrocellulose support onto which are laminated a sample pad, a conjugate pad, an analytical pad and an absorbent pad.

The sample pad to which the sample is applied is made of fibreglass and is designed to filter out blood cells, but to be permeable to antibodies.

The analytical pad includes a test line, which comprises an immobilised binding moiety specific for human IgG antibodies, e.g., a monoclonal antibody, a polyclonal antibody or a mixture of different monoclonal antibodies. An example of such an antibody is the mouse anti-human IgG monoclonal antibody B0015 produced by CTK Biotech, Inc. (CA, USA). B0015 was generated by immunising mice with human IgG and was purified from cell culture via protein A chromatography. Its isotype is IgG1 and it recognises the Fc portion of human IgG. A solution of test line antibody B0015 is prepared and coated onto the support. The analytical pad also includes a control line, which comprises an anti-chicken IgY antibody. An example of such an antibody is the goat anti-chicken IgY antibody GGHL-30A produced by Immunology Consultants Laboratory, Inc. (OR, USA). GGHL-30A was generated by immunising goats with highly purified chicken IgY h+I, collecting the antiserum and immunoaffinity purifying the antibody using a chicken IgY containing immunosorbent. A solution of the control line antibody is prepared and coated onto the support. The conjugate pad comprises dengue antigen conjugates and control conjugates. The dengue antigen conjugates comprise colloidal gold particles (nanoparticles) conjugated to a dengue antigen of interest, in this case one of the dengue antigens as described herein, which are expressed in Drosophila S2 cells. The manufacture of gold nanoparticles and their conjugation to a protein is described in Yokota S. (2010) Preparation of Colloidal Gold Particles and Conjugation to Protein A, IgG, F(ab′)₂, and Streptavidin. In: Schwartzbach S., Osafune T. (eds) Immunoelectron Microscopy. Methods in Molecular Biology (Methods and Protocols), vol 657. Humana Press, Totowa, NJ, doi.org/10.1007/978-1-60761-783-9_8. Conjugation of gold nanoparticles to a protein of interest is achieved, for example, via spontaneous adsorption of the protein to the surface of citrate-stabilised gold nanoparticles. Physical interaction between the protein and the nanoparticles depends on three phenomena: (i) ionic attraction between the negatively charged gold and the positively charged protein; (ii) hydrophobic attraction between the gold surface and hydrophobic patches on the protein and (iii) dative binding between the gold conducting electrons and free thiol groups in the protein thus forming a covalent bond. Control conjugates comprise colloidal gold particles conjugated to a chicken IgY antibody. A suitable conjugate is the CONT. CHICK product available from BBI solutions (Crumlin, U.K.).

After a suitable QC inspection, the support and laminated layers of sample pad, conjugate pad, analytical pad and absorbent pad are cut into strips of the appropriate size and then heat sealed.

In use, a body fluid (e.g. capillary whole blood) obtained from a sample taken from a subject to be tested is applied to the sample well (S) of the cassette (as shown in FIG. 2B). Approximately 5 μl of sample is loaded onto the sample pad. Running buffer (e.g., 1×PBS with 1% Tween 20) may be added to the buffer well (shown in FIG. 2B, marked as B). This causes the mixture of conjugates in the conjugate pad (and any antibodies present in the sample) to migrate along the support to the test line (marked as T in FIG. 2B) and a control line (marked as C in FIG. 2B). In the embodiment of the invention in FIG. 2B, the conjugate pad (not shown) is located between the buffer well and the sample pad. In contrast, in the embodiment of the invention in FIG. 2A, the conjugate pad is located between the analytical pad and the sample pad (i.e. the conjugate pad is more proximal to the analytical pad) and the buffer well is absent. In either orientation, any anti-dengue antibodies present in the sample (shown as green in FIG. 2A) bind to the conjugates comprising dengue antigen, before the resulting immunocomplex migrates further along the strip and is captured by the anti-human IgG antibody at the test line (which binds to the anti-dengue antibodies), thus forming a sandwich. If anti-dengue antibodies are present, captured colloidal gold particles will thus congregate at the test line, the presence of which can be visualized with the naked eye. The control conjugate is used to verify that the test has worked appropriately. When such conjugates migrate as far as the control line, they are captured by the anti-chicken IgY antibody and can again be visualized with the naked eye. Accordingly, the appearance of both the test and control lines indicates a positive result, i.e., the sample contains anti-dengue antibodies. The appearance of only the control line indicates a negative result, i.e., the sample does not contain detectable levels of anti-dengue antibodies. If neither the test line nor the control line appears, or if the test line, but not the control line appears, this indicates a failed test, which should therefore be repeated.

### Example 2A—Effect of Replacing Viral Lysate-Derived Dengue Serotype 2 (ST2) Antigen with Recombinant ST2 Envelope Antigen

This study was performed to compare the effect of replacing ST2 antigen derived from viral lysate, which has been used as the basis for an antigen-gold colloid conjugate in a commercially available dengue RDT, with a purified recombinant ST2 envelope antigen expressed in Drosophila S2 cells. Changes in RDT specificity and sensitivity were assessed against defined, well-characterised serum sample panels, as defined below.

2A-1—RDT Selection

The OnSite Dengue IgG/IgM 3.0 Combo RDT, an existing commercial lateral flow RDT manufactured by CTK Biotech (supplier catalogue number R0061C), was used as the reference test for comparison. This test contained two dengue antigens, a recombinant dengue serotype 1 (ST1) envelope antigen having the sequence as shown in SEQ ID NO: 1 and a viral lysate-derived ST2 antigen having the sequence shown in SEQ ID NO: 5; the latter antigen being derived from chimeric dengue virus licensed from the US CDC. The prototype test being compared, “Prototype 1”, was based on the commercial reference RDT, with the only alteration being the replacement of the lysate-derived ST2 antigen of SEQ ID NO: 5 with a recombinant ST2 envelope antigen having the sequence as shown in SEQ ID NO: 2 expressed in Drosophila S2 cells. To avoid any confounding effects from IgM, only the IgG component was evaluated for both tests.

2A-2—Serum Sample Panel Characterisation

The specificity serum sample panel was composed of 130 samples assessed as dengue seronegative by PRNT₅₀ assay (Timiryasova, T. M. et al., Am. J. Trop. Med. Hyg. (2013), vol. 88(5), 962-970) and/or dengue NS1 IgG ELISA, as set out in Table 1 below. JE denotes Japanese encephalitis virus and YF denotes yellow fever virus. Suitable dengue positive and negative human serum samples may be obtained from various commercial sources, for example from Access Bio, Inc. (Somerset, NJ, USA) or ABO Pharmaceuticals (San Diego, CA, USA). Alternatively, a suitable panel of serum samples may be generated simply by obtaining serum samples from a group of subjects residing in a dengue non-endemic region (e.g. the USA, mainland Europe etc.) and a group of subjects residing in a dengue endemic area (e.g. in Brazil, Colombia, Thailand, the Philippines etc.). Such samples can be classified into the different groups shown in Table 1 using the flavivirus PRNT and dengue NS1 ELISA assays as described herein.

TABLE 1
Specificity panel

| Sample | | |
|---|---|---|
| group | Definition | |
| 1 | Dengue PRNT₅₀-negative samples from subjects residing in | |
| | non-endemic region (USA), falling into 3 subgroups as | |
| | determined by as determined by JE or YF PRNT₅₀: | |
| | (i) | Flavivirus (JE and YF)-negative; |
| | (ii) | JE-positive |
| | (iii) | YF-positive |
| 2 | Dengue negative samples, assessed by dengue PRNT and | |
| | dengue NS1 IgG ELISA according to the algorithm in Figure | |
| | 1A, from subjects residing in dengue-endemic regions (Latin | |
| | America or Asia), falling into 4 subgroups as determined by | |
| | JE or YF PRNT₅₀: | |
| | (i) | JE-negative |
| | (ii) | JE-positive |
| | (iii) | YF-negative |
| | (iv) | YF-positive |

The sensitivity panel consisted of 127 dengue PRNT₅₀-positive samples, as set out in Table 2 below. Serotype identification was carried out for all virologically-confirmed dengue (VCD) samples.

TABLE 2
Sensitivity panel

| Sample | |
|---|---|
| group | Definition |
| 3 | Dengue PRNT₅₀-positive samples from subjects residing in |
| | dengue-endemic region (Latin America or Asia), further |
| | stratified according to titre of dengue-neutralising antibody: |
| | (i) PRNT₅₀-high (PRNT₅₀ > 500) |
| | (ii) PRNT₅₀-medium (PRNT₅₀ = 100 to 500) |
| | (iii) PRNT₅₀-low (PRNT₅₀ = 10 to <100) |
| 4 | Recent virologically confirmed dengue (VCD) samples, from |
| | subjects residing in dengue-endemic regions (Latin America |
| | or Asia), defined as having a prior VCD infection 1-13 |
| | months before test sample drawn |
| 5 | Remote VCD samples, from subjects residing in dengue- |
| | endemic regions (Latin America or Asia), defined as having a |
| | prior VCD infection 2-3 years before test sample drawn |

2A-3—Results and Interpretation

Results for specificity and sensitivity are shown in Table 3 and Table 4, respectively.

TABLE 3
Specificity results

| | | Reference test (OnSite | | | |
|---|---|---|---|---|---|
| | | Dengue IgG/IgM RDT) | | Prototype 1 | |
| Sample | | Specificity | (No. test | Specificity | (No. test |
| group | N | % | positive) | % | positive) |
| Total | 130 | 93.8% | (8) | 100% | (0) |
| Flavivirus | 19 | 89.5% | (2) | 100% | (0) |
| (JE & YF) (−) | | | | | |
| JE(−) | 19 | 89.5% | (2) | 100% | (0) |
| JE(+) | 15 | 100% | (0) | 100% | (0) |
| YF(−) | 23 | 73.9% | (6) | 100% | (0) |
| YF(+) | 23 | 100% | (0) | 100% | (0) |

TABLE 4
Sensitivity results

| | | Reference test (OnSite | | | |
|---|---|---|---|---|---|
| | | Dengue IgG/IgM RDT) | | Prototype 1 | |
| Sample | | Sensitivity | (No. test | Sensitivity | (No. test |
| group | N | % | positive) | % | positive) |
| Sensitivity - | 127 | 48.0% | (61) | 55.9% | (71) |
| total | | | | | |
| PRNT₅₀ high | 30 | 90.0% | (27) | 90.0% | (27) |
| PRNT₅₀ | 35 | 25.7% | (9) | 20.0% | (7) |
| medium | | | | | |
| PRNT₅₀ low | 23 | 4% | (1) | 0% | (0) |
| Prior VCD | 31 | 45.2% | (14) | 71.0% | (22) |
| recent | | | | | |
| Prior VCD | 28 | 28.6% | (8) | 53.6% | (15) |
| remote | | | | | |
| Prior VCD ST1 | 22 | 31.8% | (7) | 54.5% | (12) |
| Prior VCD ST2 | 15 | 33.3% | (5) | 80.0% | (12) |
| Prior VCD ST3 | 14 | 35.7% | (5) | 57.1% | (8) |
| Prior VCD ST4 | 8 | 62.5% | (5) | 62.5% | (5) |

This study demonstrates that substituting a recombinant dengue ST2 envelope antigen for the viral lysate-derived ST2 antigen in the existing OnSite Dengue IgG/IgM RDT is sufficient to improve test performance over the existing state of the art. The improvement in sensitivity to remote VCD is especially marked, given that prior infections might be expected to become more difficult to detect as IgG levels decrease over time.

### Example 2B

This study determined the effect on test specificity and sensitivity of introducing a dengue NS1 antigen (test prototype “F”) into an otherwise identical prototype RDT (“Prototype B”), which contained a recombinant dengue ST1 envelope antigen, having the sequence as shown in SEQ ID NO: 1, and a recombinant dengue ST2 envelope antigen, having the sequence as shown in SEQ ID NO: 2, as conjugates. The NS1 antigen was derived from dengue ST2 virus (and has the sequence shown in SEQ ID NO: 6) and was added to the Prototype B base at a final ratio of 30% of the total conjugate amount.

Specificity and sensitivity were assessed using the same sample panels as in Example 2A above. The specificity panel was augmented with additional dengue-seronegative, flavivirus-positive samples to test any effects on cross-reactivity; the other flaviviruses included were Japanese encephalitis (JE), yellow fever (YF), West Nile (WNV), and Zika viruses. The sensitivity panel was also augmented with additional dengue monotypic immune samples (ST1, 2, 3 and 4). A dengue monotypic immune sample refers to a sample which produces a positive result (antibody titre >10) to only one serotype in a dengue PRNT₉₀ assay. In contrast, a dengue multitypic immune sample refers to a sample which produces a positive result (antibody titre >10) to at least two serotypes in a dengue PRNT₉₀ assay. Sensitivity was also scored against a further panel of 200 baseline samples from the CYD14 and CYD15 clinical trials of the DENGVAXIA® vaccine (ClinicalTrials.gov numbers NCT01373281 and NCT01374516, respectively), assessed as dengue-seropositive according to the algorithm in FIG. 1A.

2B-1—Results and Interpretation

Results for specificity and sensitivity are shown in Table 6 and Table 7, respectively.

TABLE 6
Specificity results

| | | | |
|---|---|---|---|
| | | Prototype B RDT | Prototype F RDT |
| Sample panel | N | (ST1, ST2 antigens) | (ST1, ST2, NS1 antigens) |

Specificity

| | | Specificity | (No. test | Specificity | (No. test |
|---|---|---|---|---|---|
| | | % | positive) | % | positive) |
| Dengue-seronegative | 90 | 99% | (1) | 99% | (1) |
| from Example 2 | | | | | |
| (Groups 1, 2) | | | | | |

Cross-reactivity

| | | Cross- | (No. test | Cross- | (No. test |
|---|---|---|---|---|---|
| | | reactivity % | positive) | reactivity % | positive) |
| JE(+)/dengue(−) | 40 | 0% | (0) | 8% | (3) |
| YF(+)/dengue(−) | 49 | 0% | (0) | 8% | (4) |
| WNV(+)/dengue(−) | 20 | 15% | (3) | 15% | (3) |
| Zika(+)/dengue(−) | 27 | 4% | (1) | 0% | (0) |

TABLE 7
Sensitivity results

| | | Prototype B RDT | | Prototype F RDT | |
|---|---|---|---|---|---|
| | | (ST1, ST2 antigens) | | (ST1, ST2, NS1 antigens) | |
| | | Sensitivity | (No. test | Sensitivity | (No. test |
| Sample panel | N | % | positive) | % | positive) |
| Dengue-positive from | 90 | 76% | (68) | 87% | (78) |
| Example 2 (Groups 3, | | | | | |
| 4, 5) | | | | | |
| CYD14/15 baseline | 200 | 74% | (147) | 80% | (160) |
| sample panel | | | | | |
| Multitypic immune | 146 | 86% | (126) | 94% | (137) |
| Monotypic immune - | 98 | 37% | (36) | 42% | (41) |
| total | | | | | |
| Monotypic - ST 1 | 20 | 45% | (9) | 65% | (13) |
| Monotypic - ST 2 | 34 | 62% | (21) | 71% | (24) |
| Monotypic - ST 3 | 23 | 22% | (5) | 13% | (3) |
| Monotypic - ST 4 | 21 | 5% | (1) | 5% | (1) |

These results show that although introducing the NS1 antigen can confer higher RDT sensitivity, it also carries some risk of increased cross-reactivity with other flaviviruses. This could be perceived as a disadvantage. However, since the distribution of certain flaviviruses is limited to certain geographical areas (e.g., YF virus in South America and JE virus in southeast Asia), a test incorporating NS1 antigen could be utilised in those geographical areas where co-circulation of flaviviruses is less likely, thus mitigating any issues related to cross-reactivity. Utilising a test based on NS1 in this way would enable the advantages linked to increased sensitivity to be leveraged.

### Example 3

Five immunodiagnostic test devices according to the invention (B1, B1A, B1B, B1C and I1C) and one comparison immunodiagnostic test device (I1D) were prepared using a method analogous to that described in Example 1. These immunodiagnostic test devices differed from each other only in the dengue antigens present in the device, as shown in Table 8. Table 8 shows the proportion of each specific dengue antigen in B1, B1A, B1B, B1C, I1C and I1D relative to the total dengue antigen present in the device. The total concentration of dengue antigen remained the same across the immunodiagnostic test devices. In summary: B1 contains the specific Dengue antigens E1 (SEQ ID NO: 1) and E2 (SEQ ID NO: 2); B1A contains the specific Dengue antigens E1, E2 and E3 (SEQ ID NO: 3); B1B contains the specific Dengue antigens E1, E2 and E4 (SEQ ID NO: 4); B1C contains the specific Dengue antigens E1, E2, E3 and E4; I1C contains the specific Dengue antigens E1, E2, E3 and E4, as well as a further Dengue antigen (the viral lysate-derived ST2 antigen (“CDC”—SEQ ID NO: 5) from the OnSite Dengue IgG/IgM 3.0 Combo RDT referred to in Example 2 and indicated as “CDC” in the table below); and I'D contains the specific Dengue antigens E1, E3 and E4 (i.e. not E2), as well as the viral lysate-derived ST2 antigen from the OnSite Dengue IgG/IgM 3.0 Combo RDT (“CDC”—SEQ ID NO: 5).

TABLE 8
Dengue antigen composition of B1, B1A, B1B, B1C, I1C and I1D

| | Proportion of E1, E2, E3, E4 or CDC relative | | | | |
|---|---|---|---|---|---|
| Immunodiagnostic | to total dengue antigen present in device | | | | |
| test device | E1 | E2 | E3 | E4 | CDC |
| B1 | 0.5 | 0.5 | — | — | — |
| B1A | 0.3 | 0.3 | 0.4 | — | — |
| B1B | 0.3 | 0.3 | — | 0.4 | — |
| B1C | 0.3 | 0.3 | 0.2 | 0.2 | — |
| I1C | 0.2 | 0.2 | 0.2 | 0.2 | 0.2 |
| I1D | 0.3 | — | 0.2 | 0.2 | 0.3 |

The cross-reactivity, sensitivity (multitypic and monotypic), and specificity of B1, B1A, B1B and B1C were tested using the same panel of samples and method as described in Example 2B. The results of this are shown in Table 9.

TABLE 9
Cross-reactivity, sensitivity, and specificity of B1, B1A, B1B and B1C

| Parameter | Description | N | B1 | B1A | B1B | B1C | I1C | I1D |
|---|---|---|---|---|---|---|---|---|
| Cross- | JEV | 40 | 0% | 0% | 0% | 0% | 0% | 0% |
| reactivity | | | (0/40) | (0/40) | (0/40) | (0/40) | (0/40) | (0/40) |
| | YF | 49 | 4% | 0% | 2% | 0% | 0% | 0% |
| | | | (2/48) | (0/48) | (1/40) | (0/48) | (0/48) | (0/48) |
| | Zika | 27 | 0% | 0% | 0% | 0% | 0% | 0% |
| | | | (0/26) | (0/26) | (0/26) | (0/26) | (0/26) | (0/26) |
| Specificity | Group 1/2 | 90 | 99% | 100% | 99% | 100% | 100% | 99% |
| | | | (88/89) | (89/89) | (88/89) | (89/89) | (89/89) | (88/89) |
| Sensitivity | Group 3/4/5 | 90 | 83% | 86% | 86% | 88% | 90% | 87% |
| | | | (75/90) | (77/90) | (77/90) | (78/90) | (81/90) | (78/90) |
| | CYD14/15 BL | 200 | 79% | 84% | 80% | 83% | 78% | 77% |
| | | | (157/199) | (167/199) | (160/199) | (165/199) | (156/199) | (154/199) |
| | Multitypic | 146 | 92% | 96% | 92% | 95% | 92% | 92% |
| | | | (133/145) | (139/145) | (134/145) | (138/145) | (133/145) | (133/145) |
| | Monotypic | 98 | 46% | 57% | 51% | 57% | 48% | 47% |
| | (total) | | (45/98) | (56/98) | (50/98) | (56/98) | (47/98) | (46/98) |
| | Monotypic | 20 | 75% | 80% | 70% | 75% | 55% | 65% |
| | (Serotype 1) | | (15/20) | (16/20) | (14/20) | (15/20) | (11/20) | (13/20) |
| | Monotypic | 34 | 71% | 68% | 59% | 68% | 53% | 47% |
| | (Serotype 2) | | (24/34) | (23/34) | (20/34) | (23/34) | (18/34) | (16/34) |
| | Monotypic | 23 | 17% | 57% | 22% | 39% | 39% | 35% |
| | (Serotype 3) | | (4/23) | (13/23) | (5/23) | (9/23) | (9/23) | (8/23) |
| | Monotypic | 21 | 10% | 19% | 52% | 43% | 43% | 43% |
| | (Serotype 4) | | (2/21) | (4/21) | (11/21) | (9/21) | (9/21) | (9/21) |

As can be seen, including the Dengue antigens E3 and/or E4 in immunodiagnostic test devices according to the invention further increases sensitivity whilst not having a negative impact on specificity and cross-reactivity. Furthermore, including the CDC Dengue antigen together with the Dengue antigens E1, E2, E3 and E4 does not lead to any improvement in cross-reactivity or specificity, but reduces sensitivity. In particular, monotypic sensitivity is reduced. Similar results are seen when E2 is replaced by CDC.

### Example 4

A lateral flow RDT (the OnSite Dengue IgG RDT (CTK Biotech, San Diego, USA, supplier catalogue number R0065C)), was used in a retrospective analysis of serum samples from the CYD14 and CYD15 Phase III efficacy trials of DENGVAXIA®, as described in the following example. In this example, DENGVAXIA® is referred to as CYD-TDV.

### Methods

### Study Design and Participants

The phase III randomised, placebo-controlled CYD-TDV trials, CYD14 and CYD15, are described in Capeding M R, et al., 2014, Lancet; 384(9951):1358-65 and Villar L, et al., 2015, N Engl J Med., 372(2):113-23. CYD14 (NCT01373281) was conducted in children aged 2-14 years in five countries in the Asia-Pacific region (Indonesia, Malaysia, Philippines, Thailand, and Vietnam). CYD15 (NCT01374516) was conducted in children aged 9-16 years in five countries in Latin America (Brazil, Colombia, Honduras, Mexico, and Puerto Rico). The children were randomised (2:1) to receive three doses of CYD-TDV or placebo six months apart. In each study, a random subset of participants (1983/10275 [˜20%] participants from CYD14 and 2000/20869 [˜10%] from CYD15) were selected for immunogenicity analyses. Participants from this subset provided blood samples at enrolment (pre-vaccination). Those samples were used in the study described in this Example to assess vaccine efficacy outcomes and assay performance using the OnSite Dengue IgG RDT.

### Immunoassay

The OnSite Dengue IgG RDT (CTK Biotech, San Diego, USA, supplier catalogue number R0065C) is a CE-marked lateral flow immunoassay that contains the specific dengue E protein antigens E1 (which comprises the amino acid sequence SEQ ID NO: 1), E2 (which comprises the amino acid sequence SEQ ID NO: 2), E3 (which comprises the amino acid sequence SEQ ID NO: 3) and E4 (which comprises the amino acid sequence SEQ ID NO: 4). The assay qualitatively detects anti-dengue virus IgG antibodies against each of the four dengue serotypes and it identifies prior dengue infection in human serum, whole blood, or plasma samples. Testing of samples with this assay was conducted by the Global Clinical Immunology (GCI) Laboratory, Sanofi Pasteur, Swiftwater, PA, following the manufacturer's instructions. The testing personnel were blinded to the study treatment group, prior dengue exposure of the participants, and any dengue outcomes of the participants.

### Efficacy Outcomes

The study described in this Example determined CYD-TDV vaccine efficacy in participants whose pre-vaccination samples tested positive with the OnSite Dengue IgG RDT against symptomatic VCD over the active phase (up to Month 25) of CYD14 and CYD15, and against hospitalized VCD and severe dengue over the entire study period (up to Month 72). The definitions for these outcomes (symptomatic VCD, hospitalized VCD and severe dengue) are as described in DiazGranados C A et al., The Lancet Infectious diseases 2020 (https://doi.org/10.1016/S1473-3099(20)30695-2).

### Dengue Serostatus by the Reference Algorithm

Dengue serostatus for each sample was derived using previous readouts from the plaque reduction neutralisation test with 90% cut-off (PRNT₉₀), 50% cut-off (PRNT₅₀), and anti-non-structural protein 1 (NS1) IgG ELISA as described herein. An algorithm as shown in the following Table 10 was then used to categorize samples into six groups: groups one to three assigned samples as dengue seronegative, with group one being the closest possible reference to a “true” dengue seronegative; groups four to six assigned participants as dengue seropositive, with group six being closest to a “true” dengue seropositive.

TABLE 10
Algorithm for classification of reference dengue serostatus.

| | Reference test results | | | |
|---|---|---|---|---|
| | | | DV NS1 | |
| Group | PRNT₉₀¹ | PRNT₅₀¹ | IgG ELISA² | Interpretation |
| 1 | Negative | Negative | Negative | Reference seronegative |
| 2 | Negative | Positive | Negative | Reference seronegative |
| 3 | Negative | Negative | Low positive | Reference seronegative |
| 4 | Negative | Positive | Low positive | Reference seropositive |
| 5 | Negative | Any³ | High positive | Reference seropositive |
| 6 | Positive | Positive | Any³ | Reference seropositive |

¹Positive PRNT₅₀ and PRNT₉₀ were defined by a titer ≥10 (1/dil) against ≥1 dengue serotype.
²Anti-dengue NS1 IgG ELISA results were classified as negative (titer <9 EU/mL), low positive (≥9 to <50 EU/mL) and high positive (≥50 EU/mL).
³Any = positive or negative.

### Assay Performance

Sensitivity and specificity of the OnSite Dengue IgG RDT were estimated using samples classified by the dengue reference algorithm shown in Table 10. Sensitivity was calculated as the proportion of samples correctly classified as seropositive by the OnSite Dengue IgG RDT (the number of test positives/total true positives). Specificity was calculated as the proportion of samples correctly classified as seronegative by the OnSite Dengue IgG RDT (the number of test negatives/total true negatives). The positive predictive value was calculated as the number of immunoassay ‘true’ seropositives/all immunoassay seropositives while the negative predictive value was calculated as the number of immunoassay ‘true’ seronegatives/all immunoassay seronegatives.

The sensitivity of the OnSite Dengue IgG RDT was additionally determined in samples that were PRNT₉₀-positive from reference group six by immune status: monotypic with a titre of neutralizing antibodies ≥10 [1/dil] against only 1 dengue serotype; or multitypic with a titre of neutralizing antibodies ≥10 [1/dil] against ≥2 dengue serotypes. Results by immune status are also presented for five commercially-available immunoassays: the EUROIMMUN anti-dengue virus type 1-4 IgG ELISA (EUROIMMUN AG, Luebeck, Germany) (EUROIMMUN-ELISA), Panbio® Dengue IgG Indirect ELISA (Abbott, Chicago, USA) (Panbio-ELISA), TELL ME FAST™ Dengue IgG/IgM Combo Test Device (Biocan Diagnostics Inc, Vancouver, Canada) (TELL ME FAST-RDT), SD BIOLINE Dengue IgG/IgM WB (Abbott, Chicago, USA) (SD BIOLINE-RDT), and the OnSite Dengue IgG/IgM 3.0 Combo Rapid Test CE (CTK Biotech, San Diego, USA supplier catalogue number R0061C) (OnSite-IgG/IgM RDT).

### Statistical Analyses

Vaccine efficacy was assessed exclusively in participants from the immunogenicity subsets of CYD14 and CYD15 for participants who had received at least one study injection, with valid results from the reference tests, and tested positive by the OnSite Dengue IgG RDT, as CYD-TDV is not intended for use in seronegative individuals; assay performance was assessed in all included participants.

The incidence of symptomatic, hospitalised, or severe VCD was determined in terms of the number of cases per 100 person-years; the corresponding 95% confidence intervals (CIs) were calculated by the exact binomial method (Clopper-Pearson method). For efficacy outcomes from pooled CYD14 and CYD15 data, the Cox regression model was used with vaccine group and study as fixed effects. Vaccine efficacy was defined as (1-Hazard Ratio)*100 and the associated 95% CIs were calculated. Vaccine efficacy against symptomatic, hospitalised, or severe VCD was calculated overall, by age group (≥9 years, <9 years, and ≥6 years) and by study (CYD14 or CYD15).

Assay performance was calculated for the total population, by age group (≥9 years, <9 years, and ≥6 years) and by dengue immune profile (monotypic or multitypic). For sensitivity, specificity and predictive values, the 95% CI were estimated using the exact binomial method (Clopper-Pearson method).

### Results

In the CYD14 and CYD15 immunogenicity subsets, 2490/3840 (64⋅8%) samples were OnSite Dengue IgG test-positive. Baseline characteristics (age, sex, height, weight and ethnicity) were balanced between the vaccine and placebo groups for participants who tested positive with the OnSite Dengue IgG RDT (data not shown). The majority of participants who tested positive with the OnSite Dengue IgG RDT were PRNT₉₀ positive, belonging to reference group 6 (see the following Table 11).

TABLE 11
Baseline dengue serostatus in the immunogenicity
subsets of CYD14 and CYD15 by the reference
algorithm (Table 10) and OnSite Dengue IgG RDT

| | | | OnSite | | OnSite | |
|---|---|---|---|---|---|---|
| Reference | | | Dengue IgG | | Dengue IgG | |
| group | | Total | test-negative | | test-positive | |
| 1 | Reference | 851 | 789 | (92.7) | 62 | (7.3) |
| 2 | seronegative | 207 | 193 | (93.2) | 14 | (6.8) |
| 3 | | 140 | 130 | (92.9) | 10 | (7.1) |
| 4 | Reference | 58 | 51 | (87.9) | 7 | (12.1) |
| 5 | seropositive | 28 | 21 | (75.0) | 7 | (25.0) |
| 6 | | 2549 | 163 | (6.4) | 2386 | (93.6) |
| Total | | 3833 | 1347 | (35.1) | 2486 | (64.9) |

Vaccine efficacy against symptomatic VCD and hospitalised dengue in OnSite Dengue IgG test-positive participants in different age groups is as shown in the following Table 12.

TABLE 12
CYD-TDV efficacy against (A) symptomatic VCD through Month 25 and
(B) hospitalized dengue through 72 months, in the overall group
(2-16 years) and by different age cut-offs (≥9, ≥6, <9 years).

| | |
|---|---|
| n/N | |
| Density incidence (95% CI) | Vaccine efficacy |

| | | |
|---|---|---|
| CYD-TDV | Placebo | (95% CI) |

A. Symptomatic VCD through Month 25

| | | | |
|---|---|---|---|
| Overall | 15/1649 | 48/841 | 84.1 (71.6, 91.1) |

| | | | | |
|---|---|---|---|---|
| | | 0.5 (0.3, 0.8) | 2.9 (2.1, 3.8) | |
| ≥9 | years | 7/1355 | 31/681 | 88.8 (74.6, 95.1) |
| | | 0.3 (0.1, 0.5) | 2.3 (1.6, 3.3) | |
| ≥6 | years | 11/1477 | 40/753 | 86.0 (72.7, 92.8) |
| | | 0.4 (0.2, 0.7) | 2.7 (1.9, 3.7) | |
| <9 | years | 8/294 | 17/160 | 74.7 (41.4, 89.1) |
| | | 1.4 (0.6, 2.7) | 5.3 (3.1, 8.4) | |

B. Hospitalized dengue through 72 months

| | | | |
|---|---|---|---|
| Overall | 13/1649 | 22/841 | 69.2 (38.8, 84.5) |

| | | | | |
|---|---|---|---|---|
| | | 0.1 (0.1, 0.2) | 0.5 (0.3, 0.7) | |
| ≥9 | years | 7/1355 | 11/681 | 67.6 (16.4, 87.4) |
| | | <0.1 (0.0, 0.2) | 0.3 (0.1, 0.5) | |
| ≥6 | years | 8/1477 | 17/753 | 75.5 (43.2, 89.4) |
| | | <0.1 (0.0, 0.2) | 0.4 (0.2, 0.7) | |
| <9 | years | 6/294 | 11/160 | 70.6 (20.6, 89.1) |
| | | 0.4 (0.1, 0.8) | 1.2 (0.6, 2.1) | |

The sensitivity and specificity of the OnSite Dengue IgG RDT is as shown in the following Table 13.

TABLE 13
OnSite Dengue IgG RDT performance for dengue serostatus classification for
overall study population (2-16 years), and by age groups (≥9, ≥6, <9 years)

| | | | | | | Positive | Negative |
|---|---|---|---|---|---|---|---|
| Classification | Classification by reference | | | | | predictive | predictive |
| (OnSite | Sero- | Sero- | | Sensitivity | Specificity | value | value |
| IgG RDT) | positive | negative | Total | 95% CI | 95% CI | 95% CI | 95% CI |

Overall

| | | | | | | | |
|---|---|---|---|---|---|---|---|
| Sero- | 2400 | 86 | 2486 | 0.911 | 0.928 | 0.965 | 0.826 |
| positive | | | | 0.899, 0.921 | 0.912, 0.942 | 0.957, 0.972 | 0.804, 0.845 |
| Sero- | 235 | 1112 | 1347 | | | | |
| negative | | | | | | | |
| Total | 2635 | 1198 | 3833 | | | | |

≥9 years

| | | | | | | | |
|---|---|---|---|---|---|---|---|
| Sero- | 2013 | 22 | 2035 | 0.924 | 0.966 | 0.989 | 0.790 |
| positive | | | | 0.912, 0.935 | 0.949, 0.978 | 0.984, 0.993 | 0.760, 0.818 |
| Sero- | 165 | 622 | 787 | | | | |
| negative | | | | | | | |
| Total | 2178 | 644 | 2822 | | | | |

≥6 years

| | | | | | | | |
|---|---|---|---|---|---|---|---|
| Sero- | 2197 | 31 | 2228 | 0.918 | 0.961 | 0.986 | 0.796 |
| positive | | | | 0.907, 0.929 | 0.945, 0.973 | 0.980, 0.991 | 0.770, 0.822 |
| Sero- | 195 | 763 | 958 | | | | |
| negative | | | | | | | |
| Total | 2392 | 794 | 3186 | | | | |

<9 years

| | | | | | | | |
|---|---|---|---|---|---|---|---|
| Sero- | 387 | 64 | 451 | 0.847 | 0.884 | 0.858 | 0.875 |
| positive | | | | 0.810, 0.879 | 0.855, 0.910 | 0.822, 0.889 | 0.845. 0.901 |
| Sero- | 70 | 490 | 560 | | | | |
| negative | | | | | | | |
| Total | 457 | 554 | 1011 | | | | |

The OnSite Dengue IgG RDT exhibited very high sensitivity (91⋅1%; 95% CI 89⋅9, 92⋅1) and a high specificity (92⋅8%; 95% CI 91⋅2, 94⋅2) for the entire study population (aged 2-16 years). Among the different age groups, both sensitivity and specificity were comparable for participants ≥9 and ≥6 years of age (sensitivity: 92⋅4% and 91⋅8%; specificity: 96⋅6% and 96⋅1%, for the two age groups, respectively). The positive and negative predictive values were comparable for participants ≥9 and ≥6 years of age (positive predictive values: 0⋅989 and 0⋅986 and negative predictive values: 0⋅790 and 0⋅796 for the two age groups respectively). For participants who had a monotypic immune profile, sensitivity was 82⋅9% with the OnSite Dengue IgG RDT, ranging from 75⋅2% to 87⋅5% for each of the dengue serotypes (see Table 14). This was comparable to the sensitivity observed with the Panbio-ELISA (84⋅0%), higher than that with the EUROIMMUN-ELISA (74⋅4%), and substantially higher than for the three commercial IgM-A/IgG RDTs (range 12⋅1-44⋅6%) (Table 14).

TABLE 14
Sensitivity of OnSite Dengue IgG RDT and five commercial serotests in
PRNT₉₀-positive participants from reference group 6, by immune profile and serotype

| | | | | | OnSite | | SD | | |
|---|---|---|---|---|---|---|---|---|---|
| | | | | | IgG/IgM | TELL ME | BIOLINE- | EUROIMMUN- | Panbio- |
| | OnSite Dengue IgG RDT | | | | RDT | FAST-RDT | RDT | ELISA | ELISA |
| | Test- | Test- | | Sensitivity, % | Sensitivity, % | Sensitivity, | Sensitivity, | Sensitivity, % | Sensitivity, |
| | negative | positive | Total | (95% CI) | (95% CI) | % (95% CI) | % (95% CI) | (95% CI) | % (95% CI) |
| All | 160 | 2380 | 2540 | 93.7 | 49.3 | 54.2 | 73.1 | 91.7 | 95.0 |
| | | | | (92.7, 94.6) | (47.4, 51.2) | (52.2, 56.1) | (71.4, 74.9) | (90.5, 92.7) | (94.1, 95.8) |
| Multitypic | 34 | 1768 | 1802 | 98.1 | 64.5 | 67.3 | 84.8 | 98.7 | 99.5 |
| | | | | (97.4, 98.7) | (62.3, 66.7) | (65.1, 69.4) | (83.1, 86.5) | (98.1, 99.2) | (99.1, 99.8) |
| Monotypic | 126 | 612 | 738 | 82.9 | 12.1 | 22.2 | 44.6 | 74.4 | 84.0 |
| | | | | (80.0, 85.6) | (9.9, 14.7) | (19.3, 25.4) | (41.0, 48.3) | (71.1, 77.5) | (81.2, 86.6) |
| Serotype 1 | 26 | 127 | 153 | 83.0 | 17.7 | 80.4 | 39.7 | 67.8 | 92.3 |
| | | | | (76.1, 88.6) | (12.1, 24.6) | (73.3, 86.3) | (32.0, 47.9) | (59.7, 75.1) | (86.9, 96.0) |
| Serotype 2 | 43 | 300 | 343 | 87.5 | 15.3 | 8.3 | 59.6 | 86.8 | 83.1 |
| | | | | (83.5, 90.8) | (11.7, 19.5) | (5.6, 11.7) | (54.2, 64.8) | (82.8, 90.2) | (78.8, 86.9) |
| Serotype 3 | 32 | 109 | 141 | 77.3 | 4.8 | 7.7 | 30.3 | 70.4 | 97.2 |
| | | | | (69.5, 83.9) | (2.0, 9.7) | (3.9, 13.3) | (22.9, 38.5) | (62.2, 77.8) | (92.9, 99.2) |
| Serotype 4 | 25 | 76 | 101 | 75.2 | 3.6 | 1.0 | 21.6 | 48.0 | 55.9 |
| | | | | (65.7, 83.3) | (1.0, 9.0) | (0.0, 5.2) | (14.0, 30.8) | (38.0, 58.2) | (45.7, 65.7) |

### DISCUSSION

In this Example, the new OnSite Dengue IgG RDT was used to retrospectively determine baseline dengue serostatus of participants from the pivotal CYD-TDV efficacy studies. In those aged 2-16 years and who were determined to be baseline dengue seropositive by the assay, the vaccine efficacy of CYD-TDV demonstrated robust protection against symptomatic VCD over two years (vaccine efficacy 84.1%), and hospitalised VCD over six years (vaccine efficacy 69.2%). This was also observed for each of the age groups, including those ≥6 years with a vaccine efficacy of 86.0% and 75.5% for symptomatic and hospitalised VCD, respectively.

While high test specificity would ensure that the risk to seronegative persons is minimised, high sensitivity is important to ensure that the maximum number of eligible individuals benefit. In the indicated age group for CYD-TDV, ≥9 years, the OnSite Dengue IgG RDT demonstrated high specificity (97%) and high sensitivity (92%); similar results were observed in the ≥6 years age group.

Modelling of pre-vaccination screening strategies has illustrated the potential benefits of a screen-and-vaccinate strategy, and has shown that such strategies are improved by the increasing sensitivity of RDTs. The OnSite Dengue IgG RDT demonstrated good sensitivity for those with evidence for infection by only one dengue serotype (monotypic; 82⋅9%) and was highly sensitive for those with multitypic profiles (98⋅1%). The sensitivity for monotypic dengue was balanced across the four serotypes and comparable or superior to that seen with previously evaluated dengue IgG ELISAs. However, the OnSite Dengue IgG RDT, being more convenient and easier to implement than ELISAs, would offer the additional advantage of screening at the point-of-care. The sensitivity for monotypic dengue of the OnSite Dengue IgG RDT was substantially higher than that for current commercially available RDTs. As monotypic dengue is a proxy for having experienced one prior dengue infection, the identification of monotypic individuals is particularly important in reducing severe dengue cases as these individuals have an increased risk of severe dengue infection upon secondary infection (Katzelnick L C et al., Science 2017; 358(6365): 929-32).

The selection of an RDT for point-of-care pre-vaccination screening depends on a number of factors including accuracy and ease of use. As well as offering high specificity and sensitivity as shown above, the OnSite Dengue IgG RDT can be conducted within 25 minutes (unlike ELISAs), and therefore can facilitate convenient testing at the time of vaccination (e.g. within a single clinic visit), thereby increasing compliance and uptake of dengue vaccination.

Whilst the present invention has been described and illustrated with reference to particular embodiments, it will be appreciated by those of ordinary skill in the art that the invention lends itself to many different variations not specifically illustrated herein. By way of example only, certain possible variations will now be described.

Where in the foregoing description, integers or elements are mentioned which have known, obvious or foreseeable equivalents, then such equivalents are herein incorporated as if individually set forth. Reference should be made to the claims for determining the true scope of the present invention, which should be construed so as to encompass any such equivalents. It will also be appreciated by the reader that integers or features of the invention that are described as preferable, advantageous, convenient or the like are optional and do not limit the scope of the independent claims. Moreover, it is to be understood that such optional integers or features, whilst of possible benefit in some embodiments of the invention, may not be desirable, and may therefore be absent, in other embodiments.

1. An immunodiagnostic test device for the detection of anti-dengue virus antibodies comprising a first dengue antigen and a second dengue antigen, wherein said first dengue antigen comprises a polypeptide having at least 90% sequence identity to SEQ ID NO. 1 and said second dengue antigen comprises a polypeptide having the sequence of SEQ ID NO. 2 or a polypeptide having a sequence which has at least 1 and no more than 4 amino acid substitutions with respect to the sequence of SEQ ID NO. 2.

2. The immunodiagnostic test device of claim 1, wherein said polypeptide of said second dengue antigen which has at least 1 and no more than 4 amino acid substitutions with respect to the sequence of SEQ ID NO. 2 does not comprise an amino acid substitution with respect to SEQ ID NO. 2 at the positions in the polypeptide which correspond to positions 5, 52, 119, 125 and 202 of SEQ ID NO. 2.

3. The immunodiagnostic test device of claim 1, wherein said polypeptide of said first dengue antigen does not comprise an amino acid substitution with respect to SEQ ID NO. 1 at the positions in the polypeptide which correspond to positions 97 to 109 of SEQ ID NO. 1, and wherein said polypeptide of said second dengue antigen which has at least 1 no more than 4 amino acid substitutions with respect to the sequence of SEQ ID NO. 2, does not comprise an amino acid substitution with respect to SEQ ID NO. 2 at the positions in the polypeptide which correspond to positions 97 to 109 of SEQ ID NO. 2.

4. The immunodiagnostic test device of claim 1, wherein said device further comprises a third dengue antigen and a fourth dengue antigen, wherein said third dengue antigen comprises a polypeptide having at least 90% sequence identity to SEQ ID NO. 3 and said fourth dengue antigen comprises a polypeptide having at least 90% sequence identity to SEQ ID NO. 4.

5. The immunodiagnostic test device of claim 4, wherein said polypeptide of said third dengue antigen does not comprise an amino acid substitution with respect to SEQ ID NO. 3 at the positions in the polypeptide which corresponds to positions 98 to 110 of SEQ ID NO. 3, and wherein said polypeptide of said fourth dengue antigen does not comprise an amino acid substitution with respect to SEQ ID NO. 4 at the positions in the polypeptide which correspond to positions 97 to 109 of SEQ ID NO. 4.

6. The immunodiagnostic test device of claim 4, wherein said polypeptides of said first, third and fourth dengue antigens have the sequence of SEQ ID NO. 1, SEQ ID NO. 3 and SEQ ID NO. 4 respectively, or said polypeptides of said first, third and fourth dengue antigens have sequences which have at least 1 and no more than 4 amino acid substitutions with respect to the sequences of SEQ ID NO. 1, SEQ ID NO. 3 and SEQ ID NO. 4 respectively.

7. The immunodiagnostic test device of claim 4, wherein said polypeptides of said first, third and fourth dengue antigens have the sequence of SEQ ID NO. 1, SEQ ID NO. 3 and SEQ ID NO. 4 respectively or said polypeptides of said first, third and fourth dengue antigens have sequences which have at least 1 and no more than 4 amino acid substitutions with respect to the sequences of SEQ ID NO. 1, SEQ ID NO. 3 and SEQ ID NO. 4 respectively.

8. The immunodiagnostic test device of claim 4, wherein said polypeptides of said first, second, third and fourth antigens have the sequence of SEQ ID NO. 1, SEQ ID NO. 2, SEQ ID NO. 3 and SEQ ID NO. 4 respectively.

9. The immunodiagnostic test device of claim 4, wherein said test device comprises no other dengue antigens.

10. The immunodiagnostic test device of claim 1, wherein at least one of said dengue antigens comprises at least one epitope which is capable of binding to said anti-dengue virus antibodies, optionally wherein said anti-dengue virus antibodies are IgG antibodies.

11. (canceled)

12. The immunodiagnostic test device of claim 1, wherein said device comprises a support, a sample pad, a conjugate pad, an analytical pad, an absorbent pad, at least one detection line and at least one quality control line.

13. The immunodiagnostic test device of claim 12, wherein said conjugate pad comprises said dengue antigens and wherein each of said dengue antigens are conjugated to a detectable moiety, optionally wherein said detectable moiety is a gold colloid.

14. (canceled)

15. The immunodiagnostic test device of claim 12, wherein at least one capture moiety is immobilised at said at least one detection line.

16. The immunodiagnostic test device of claim 15, wherein said at least one capture moiety is an anti-human IgG antibody.

17. (canceled)

18. (canceled)

19. A method for determining a dengue infection in a human subject, the method comprising:
a. providing a first dengue antigen and a second dengue antigen, wherein said first and second dengue antigens are as defined in claim 1 and wherein said dengue antigens are conjugated to a detectable moiety;
b. contacting the dengue antigens with a biological sample obtained from the subject, the biological sample optionally containing at least one binding partner capable of binding to at least one of the antigens; and
c. if said sample contains said at least one binding partner, detecting the presence of a complex that forms between at least one of the dengue antigen-detectable moiety conjugates and the at least one binding partner, which presence indicates a dengue virus infection in the subject.

20. The method of claim 19, wherein said step (a) further comprises providing a third dengue antigen and a fourth dengue antigen, wherein said third dengue antigen comprises a polypeptide having at least 90% sequence identity to SEQ ID NO. 3 and said fourth dengue antigen comprises a polypeptide having at least 90% sequence identity to SEQ ID NO. 4, and wherein said third dengue antigen and said fourth dengue antigen are conjugated to a detectable moiety.

21.-25. (canceled)

26. The method according to claim 19, wherein the dengue infection is a prior dengue infection.

27. The method according to claim 19, wherein said step (c) of detecting the presence of a complex that forms between at least one of the dengue antigen-detectable moiety conjugates and the at least one binding partner comprises contacting the complex with a molecule capable of binding to the at least one binding partner.

28. The method according to claim 27, wherein said molecule capable of binding to the at least one binding partner is an antibody directed against human antibodies, optionally wherein the antibody directed against human antibodies is an antibody specific for human IgG antibodies.

29.-32. (canceled)

33. A method of vaccinating a human subject against dengue,
comprising the step of administering a dengue vaccine to said subject, wherein said human subject has previously been shown to be dengue seropositive using the immunodiagnostic test device of claim 1.

34. (canceled)
